(19) United States
(12) Patent Application Publication
(10) Pub. No.: US 2021/0241916 A1
(43) Pub. Date: Aug. 5, 2021

(54) FORECASTING AND EXPLAINING USER HEALTH METRICS

(71) Applicant: INFORMED DATA SYSTEMS INC. d/b/a ONE DROP, New York, NY (US)

(72) Inventors: Ydo Wexler, Haifa (IL); Daniel R. Goldner, Minnetonka, MN (US); Jeffrey Dachis, Brooklyn, NY (US)

(21) Appl. No.: 17/167,795

(22) Filed: Feb. 4, 2021

Related U.S. Application Data
(60) Provisional application No. 62/970,282, filed on Feb. 5, 2020.

(51) Int. Cl. G16H 50/30; G16H 10/60; A61B 5/021; A61B 5/145; A61B 5/00

(52) U.S. Cl. CPC G16H 50/30; G16H 10/60; A61B 5/6802; A61B 5/14532; A61B 5/6898; A61B 5/021

(57) ABSTRACT

Systems and methods for forecasting and/or explaining health metrics are provided. In some embodiments, for example, a method for informing a user of at least one health factor contributing to a predicted health metric comprises receiving health data of the user, and calculating a plurality of features based on the health data. The features can be categorized into a plurality of feature groups. The method further includes generating a prediction of a health metric of the user by inputting the features into a machine learning model. The method can also include identifying at least one contributing health factor by selecting at least one feature group that provides a threshold contribution to the prediction, and determining a health factor associated with the at least one feature group. The method can include outputting a notification for the user including the prediction and the at least one contributing health factor.

### CROSS-REFERENCE TO RELATED APPLICATION(S)

The present application claims the benefit of U.S. Provisional Patent Application No. 62/970,282, filed on Feb. 5, 2020, which is incorporated by reference herein in its entirety.

### INCORPORATION BY REFERENCE OF APPLICATIONS AND PATENTS

The following commonly assigned U.S. Patent Applications are incorporated herein by reference in their entireties:

U.S. Patent Application Publication No. 2020/0077931, entitled “FORECASTING BLOOD GLUCOSE CONCENTRATION.”

U.S. Patent Application Publication No. 2020/0375549, entitled “SYSTEMS FOR BIOMONITORING AND BLOOD GLUCOSE FORECASTING, AND ASSOCIATED METHODS.”

### TECHNICAL FIELD

This disclosure relates generally to data processing and, in particular, to systems and methods for forecasting and/or explaining health metrics of a user.

### BACKGROUND

Health of a human being may be assessed using various metrics, such as weight, blood pressure, blood glucose levels, or cholesterol levels. These metrics may be indicative of medical conditions, such as hypertension, diabetes, or prediabetes. Various programs exist to assist individuals in making behavioral changes to improve health metrics, such as by lowering their blood pressure, blood glucose concentration, or weight. However, significant and lasting improvements in these metrics may take weeks or even months to achieve. During that time, individuals may not know if their efforts are likely to succeed, and that uncertainty may reduce their motivation to make long-term behavioral changes. Additionally, it may be difficult for individuals to determine what kinds of behavioral changes are likely to produce significant improvements in health metrics.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments depicted, but are for explanation and understanding only.

FIG. 1 is a schematic diagram of a computing environment in which a biomonitoring and forecasting system operates, in accordance with embodiments of the present technology.

FIG. 2A illustrates a training/retraining system configured in accordance with embodiments of the present technology.

FIG. 2B illustrates a prediction generation system configured in accordance with embodiments of the present technology.

FIGS. 3A-3C are flow charts illustrating a process for forecasting a health metric of a user, in accordance with embodiments of the present technology.

FIG. 4 illustrates a representative example of a user interface including a graphical representation of a support message configured in accordance with embodiments of the present technology

FIG. 5 is a flowchart illustrating a method for forecasting a health metric of a user, in accordance with embodiments of the present technology.

FIG. 6 is a schematic block diagram of a computing system or device configured in accordance with embodiments of the present technology.

### DETAILED DESCRIPTION

The present technology generally relates to systems and methods for forecasting, explaining, and/or interpreting various health metrics, including, for example, blood pressure, blood glucose values (e.g., long term average blood glucose values), weight values, and/or other measurements of a user's health state. In some embodiments, for example, a method for forecasting a health metric of a user includes receiving health data of the user (e.g., blood pressure data, blood glucose data, heart rate data, weight data, etc.), and determining a plurality of features from the health data. The features can be categorized into a plurality of feature groups, each feature group associated with a health factor (e.g., blood pressure, blood glucose, weight, diet, activity, demographics, etc.). The method can further include generating a prediction of the health metric by inputting the features into a prediction model (e.g., a machine learning model trained on data from many different individuals). The prediction can provide an estimated probability that the user's health metric will achieve a target value and/or be within a target range at a future time period (e.g., one, two, three, four, five, six, or more months in the future).

The present technology can also assist the user in making lifestyle changes by predicting which health factors are likely to significantly influence the health metric. In some embodiments, the method further includes determining a contribution of each feature group to the prediction of the health metric (e.g., using an explanation model and/or attribution algorithm). Subsequently, the method can include outputting a notification to the user including the prediction and at least one health factor associated with the feature group(s) determined to have contributed to the prediction (e.g., the feature group having the greatest contribution to the prediction). The present technology can provide accurate, long-term forecasts of many different types of health metrics, which can be beneficial in helping users monitor and/or predict their progress towards their health goals. The present technology can also guide, motivate, and/or otherwise assist the user in improving their health by predicting which health factors are most likely to contribute to significant and lasting changes in health metrics.

As used herein, the term “health metrics” encompasses any measure relevant to a user's health state, including, but not limited to, measurements and/or estimates of any of the following: blood pressure, blood glucose (e.g., blood glucose levels and/or time-in-range values), weight, body mass index (BMI), waist circumference, body fat percentage, cholesterol, triglycerides, heart rate, respiratory rate, body temperature, gases (e.g., oxygen, carbon dioxide), electrolytes (e.g., bicarbonate, potassium, sodium, magnesium, chloride, lactic acid), blood urea nitrogen (BUN), creatinine, ketones, alcohols, neurotransmitters, amino acids, hormones, disease biomarkers (e.g., cancer biomarkers, cardiovascular disease biomarkers), and/or combinations thereof. Such health metrics may also be interchangeably referred to herein as “metabolic metric data,” “metabolic health metrics,” “metabolic metrics,” “metric data,” and/or “metrics.”

The headings provided herein are for convenience only and are not intended to limit or interpret the scope or meaning of the technology.

### Overview of Technology

A person's health can be assessed using various health metrics, such as weight, blood pressure, blood glucose levels, cholesterol levels, and many others. One or more of these metrics may be indicative of health conditions (e.g., hypertension, diabetes, prediabetes, etc.) that would benefit from medical treatment, lifestyle changes, etc. For example, diabetes mellitus (DM) is a group of metabolic disorders characterized by high blood glucose levels over a prolonged period. Typical symptoms of such conditions include frequent urination, increased thirst, increased hunger, etc. If left untreated, diabetes can cause many complications. There are three main types of diabetes: Type 1 diabetes, Type 2 diabetes, and gestational diabetes. Type 1 diabetes results from the pancreas' failure to produce enough insulin. In Type 2 diabetes, cells fail to respond to insulin properly. Gestational diabetes occurs when pregnant women without a previous history of diabetes develop high blood glucose levels.

Diabetes affects a significant percentage of the world's population. Timely and proper diagnoses and treatment are essential to maintaining a relatively healthy lifestyle for individuals with diabetes. Application of treatment typically relies on accurate determination of glucose concentration in the blood of an individual at a present time and/or in the future. However, conventional blood glucose monitoring systems may be unable to provide real-time analytics, personalized analytics, or blood glucose concentration forecasting, or may not provide such information in a rapid, reliable, and accurate manner.

Prediabetes is a serious medical condition characterized by higher than normal blood sugar levels that are not yet high enough to be diagnosed as Type 2 diabetes. With prediabetes, the cells in the body do not respond normally to insulin, which causes the pancreas to produce more insulin to try to elicit the appropriate response. Eventually, however, the pancreas is unable to keep up and blood glucose levels rise, causing prediabetes and, later on, Type 2 diabetes. Typically, prediabetes may go undetected until occurrence of serious health problems, e.g., Type 2 diabetes. There are several risk factors that are associated with prediabetes: being overweight, being 45 years or older, having a family history of Type 2 diabetes, being physically inactive, having gestational diabetes (diabetes during pregnancy), giving birth to a baby who weighed more than 9 pounds, having polycystic ovary syndrome, and others. Various lifestyle, diet, habit, and/or other changes may be helpful in dealing with prediabetes and/or eventual Type 2 diabetes.

As stated above, assessment of whether a person has diabetes and/or prediabetes may begin with measuring the person's blood glucose concentration, which is typically measured in milligrams per deciliter (mg/dL). As blood glucose values can vary during the day, especially in individuals with diabetes, average blood glucose values are generally used to assess long term progress. Average blood glucose values can be estimated directly as the average of many instantaneous measurements over a time period (e.g., a month or more). Alternatively or in combination, HbA1c, a measure of the amount of glycation of red blood cells, which is generally proportional to average blood glucose concentration, is also commonly used.

Hypertension, which is sometimes referred to as high blood pressure, is a long-term medical condition where the blood pressure in an individual's arteries is constantly elevated. Long-term high blood pressure is considered a major risk factor for coronary artery disease, stroke, heart failure, atrial fibrillation, peripheral arterial disease, vision loss, chronic kidney disease, dementia, and other serious medical conditions. There are several factors that increase likelihood of high blood pressure. Some of these include lifestyle factors, e.g., excess use of salt in a diet, excess body weight, smoking, alcohol use, etc. Others include identifiable causes, such as serious medical conditions, e.g., chronic kidney disease, narrowing of the kidney arteries, an endocrine disorder, etc.

Blood pressure can be measured using systolic and diastolic pressures corresponding to maximum and minimum pressures, respectively. For most adults, normal resting blood pressure is in a range of 100-130 millimeters mercury (mmHg) systolic and 60-80 mmHg diastolic. Consistent measurements of blood pressure above these ranges may be considered high blood pressure. Normal and high blood pressure ranges for children may be different.

Treatment of diseases and/or conditions such as high blood pressure, diabetes or prediabetes, etc., may prevent or mitigate detrimental effects on the person's health and/or reduce the risk of developing more serious medical conditions. In some embodiments, the individual may respond to a combination of medicine, lifestyle changes, or both. Lifestyle changes may include, for example, weight loss, physical exercise, decreased salt intake, decreased alcohol intake, a healthier diet, etc. However, it may take weeks or months before meaningful changes become evident (e.g., based on heart function data, etc.), and individuals may lose motivation if they are uncertain whether their lifestyle changes are having any meaningful impact on their health. Accordingly, there is a need for improved systems and methods that forecast future changes in the user's health metrics and/or provide explanations for changes in the health metrics to guide users in reaching their health goals.

In some embodiments, for example, the present technology relates to a computer-implemented method for forecasting and interpreting one or more metabolic health metrics for a user. The method may include determining features (e.g., input data parameters) for training a forecasting data model, training the model, generating predictions of one or more of health metrics (e.g., average blood pressure, average blood glucose, weight, etc.), determining confidence intervals for the predictions, determining a probability of reaching a goal (e.g., a target value of one or more metabolic health metric), combining forecasting data, confidence intervals and the determined probability for display to a user, and interpreting the forecasted data, e.g., to provide feedback to the user.

In some embodiments, the present technology provides a computing system and/or framework for performing such determining, forecasting and/or interpretation of health metric data related to the user. The health metric data may include, for example, blood pressure data, blood glucose concentration (e.g., average, standalone, etc.) data, weight data, etc. and/or any combination thereof.

Non-transitory computer program products (i.e., physically embodied computer program products) are also described that store instructions, which when executed by one or more data processors of one or more computing systems, causes at least one data processor to perform the operations disclosed herein. Similarly, computer systems are also described that may include one or more data processors and memory coupled to the one or more data processors. The memory may temporarily or permanently store instructions that cause at least one processor to perform one or more of the operations described herein. In addition, methods may be implemented by one or more data processors either within a single computing system or distributed among two or more computing systems. Such computing systems may be connected and may exchange data and/or commands or other instructions or the like via one or more connections, including but not limited to a connection over a network (e.g., the Internet, a wireless wide area network, a local area network, a wide area network, a wired network, or the like), via a direct connection between one or more of the multiple computing systems, etc.

The details of one or more variations of the present technology described herein are set forth in the accompanying drawings and the description below. Other features and advantages of the subject matter described herein will be apparent from the description and drawings, and from the claims.

### Systems for Biomonitoring and Forecasting

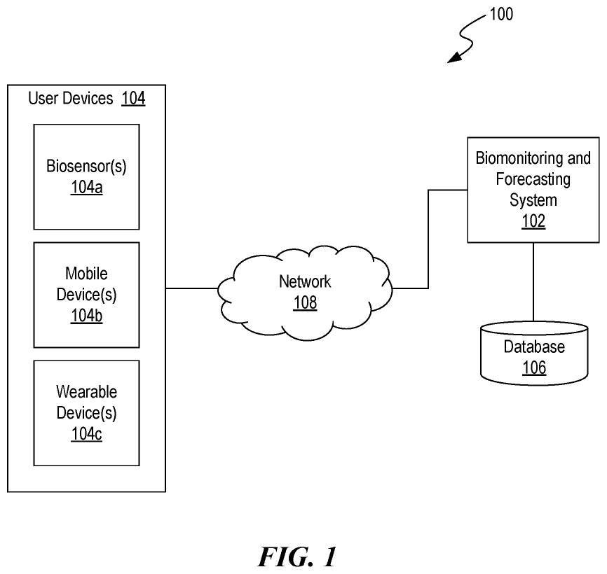

FIG. 1 is a schematic diagram of a computing environment 100 in which a biomonitoring and forecasting system 102 (“system 102”) operates, in accordance with embodiments of the present technology. As shown in FIG. 1, the system 102 is operably coupled to one or more user devices 104 via a network 108. The system 102 is also operably coupled to at least one database or storage component 106 (“database 106”). The system 102 can be configured to perform input, determination, analysis, forecasting, and/or interpretation of a user's health metrics, such as blood glucose, blood pressure, weight, triglycerides, cholesterol, BMI, waist circumference, etc. In some embodiments, the system 102 includes processors, memory, and/or other software and/or hardware components configured to implement the various methods described herein. For example, the system 102 can be or include a forecasting and/or analysis engine, and/or a server, which can be collectively configured to generate predictions of a user's health metrics. The system 102 can receive input data (e.g., blood glucose data, blood pressure data, and/or other data described herein) and perform monitoring, processing, analysis, forecasting, interpretation, etc. of the input data in order to generate the predictions. The system 102 can also be configured to output notifications, recommendations, and/or other information to the user based on the predicted health metrics. Additional details of the operations performed by the system 102 are provided further below.

In some embodiments, the system 102 receives input data from one or more user devices 104. The user devices 104 can be any device associated with a user or patient, and can be used to obtain health metric data (e.g., blood pressure data, blood glucose data, weight data) and/or any other relevant input data (e.g., food data, medication data, physical activity data, sleep data, demographic data, etc.) relating to the user and/or any other users (e.g., appropriately anonymized patient data). In the illustrated embodiment, for example, the user devices 104 include at least one biosensor 104a (e.g., a sensor for obtaining user data), at least one mobile device 104b (e.g., a smartphone or tablet computer), and, optionally, at least one wearable device 104c (e.g., a smartwatch or fitness tracker). In other embodiments, however, one or more of the devices 104a-c can be omitted and/or other types of user devices can be included (e.g., computing devices such as personal computers, laptop computers, etc.). Additionally, although FIG. 1 illustrates the biosensor(s) 104a as being separate from the other user devices 104, in other embodiments the biosensor(s) 104a can be incorporated into another user device 104 (e.g., in a smartwatch, fitness tracker, smartphone, etc.).

The biosensor(s) 104a can include any device capable of obtaining user health data, such as implanted sensors, non-implanted sensors, invasive sensors, minimally invasive sensors, non-invasive sensors, wearable sensors, etc. In some embodiments, the biosensor(s) 104a are configured to monitor and/or analyze one or more of the following: body fluids (e.g., blood, interstitial fluid, sweat, etc.), tissue (e.g., optical characteristics of body structures, anatomical features, skin, etc.), and/or vitals (e.g., heat rate, blood pressure, etc.). For example, the biosensor(s) 104a can include at least one blood pressure sensor configured to obtain blood pressure measurements of the user, using any suitable technique known to those of skill in the art. As another example, the biosensor(s) 104a can include at least one sensor configured to analyze body fluids (e.g., blood, interstitial fluid, sweat, etc.) from the user to determine levels of an analyte in the body fluid, such as blood glucose levels. In some embodiments, the biosensor(s) 104a can include at least blood glucose sensor, such as a continuous glucose monitoring (CGM) sensor.

The monitoring performed by the biosensor(s) 104a can be performed continuously, periodically, or a suitable combination thereof. For example, the biosensor(s) 104a can obtain user data at any suitable time interval, such as every minute, 2 minutes, 5 minutes, 10 minutes, 15 minutes, 20 minutes, 30 minutes, 60 minutes, 2 hours, 12 hours, 24 hours, 48 hours, etc. In some embodiments, the time interval is within a range from 5 minutes to 10 minutes. Optionally, the biosensor(s) 104a can include other capabilities, such as processing, transmitting, receiving, and/or other computing capabilities.

The user devices 104 can also include one or more devices that allow for entry of additional types of data, such as meal or nutrition data (e.g., number of meals; timing of meals; number of calories; amount of carbohydrates, fats, sugars, etc.), medical history or other data (e.g., current and/or previous weight, height, BMI, age, sleeping patterns, medical conditions, cholesterol levels, diabetes type, family history, user health history, diagnoses, blood pressure, etc.), physical activity and/or exercise data (e.g., time and/or duration of activity; activity type such as walking, running, swimming; strenuousness of the activity such as low, moderate, high; etc.), personal data (e.g., name, gender, demographics, social network information, etc.), medication data (e.g., timing and/or dosages of medications such as insulin, prescription and/or non-prescription medications taken), blood glucose data (e.g., current and/or previous blood glucose measurements, current and/or previous HbA1c data values), and/or any other data, and/or any combination thereof.

In some embodiments, one or more of the user devices 104 can be configured to obtain other physiological data of the user, such as cardiovascular data, respiratory data, body temperature data (e.g., skin temperature data), sleep data, stress level data (e.g., cortisol and/or other chemical indicators of stress levels), a1c data, biomarker data (e.g., for other diseases or conditions), energy consumption data, oxygen consumption data, and/or data of any other suitable physiological parameters. For example, cardiovascular data can include any physiological parameter related to the user's cardiovascular health, such as blood pressure data, heart rate data, arrhythmia event data (if any), pacemaker data, etc. In some embodiments, the cardiovascular data can be the “most recent” data, e.g., data taken within the last minute, 2 minutes, 5 minutes, 10 minutes, 15 minutes, 20 minutes, 30 minutes, 60 minutes, 2 hours, etc. For example, the blood pressure data can include systolic and/or diastolic blood pressure measurement(s) of the user.

In some embodiments, some or all of the user devices 104 are configured to continuously obtain any of the above data (including blood pressure data, blood glucose data, health data, etc.) from the user over a particular time period (e.g., hours, days, weeks, months, years). For example, data can be obtained at a predetermined time interval (e.g., once every minute, 2 minutes, 5 minutes, 10 minutes, 15 minutes, 20 minutes, 30 minutes, 60 minutes, 2 hours, 12 hours, 24 hours, 48 hours, etc.), at random time intervals, or combinations thereof. The time interval for data collection can be set by the user, by another user (e.g., a physician), by the system 102, or by the user device 104 itself (e.g., as part of an automated data collection program). The user device 104 can obtain the data automatically or semi-automatically (e.g., by automatically prompting the user to provide such data at a particular time), or from manual input by the user (e.g., without prompts from the user device 104). In some embodiments, data may be provided to the system 102 at predetermined time intervals (e.g., once every minute, 2 minutes, 5 minutes, 10 minutes, 15 minutes, 20 minutes, 30 minutes, 60 minutes, 2 hours, etc.), continuously, in real-time, upon receiving a query, manually, automatically (e.g., upon detection of new data), semi-automatically, etc. The time interval at which the user device 104 obtains data may or may not be the same as the time interval at which the user device 104 transmits the data to the system 102.

The user devices 104 can obtain any of the above data in various ways, such as using one or more of the following components: a microphone (e.g., a separate microphone or a microphone imbedded in the device), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, a sensor (e.g., a sensor included in or operably coupled to the user device 104), and/or any other device. The data obtained by the user devices 104 can include metadata, structured content data, unstructured content data, embedded data, nested data, hard disk data, memory card data, cellular telephone memory data, smartphone memory data, main memory images and/or data, forensic containers, zip files, files, memory images, and/or any other data/information. The data can be in various formats, such as text, numerical, alpha-numerical, hierarchically arranged data, table data, email messages, text files, video, audio, graphics, etc. Optionally, any of the above data can be filtered, smoothed, augmented, annotated, or otherwise processed (e.g., by the user devices 104 and/or the system 102) before being used for analysis and/or forecasting, as described in greater detail below. Alternatively, the user devices 104 can transmit raw data directly to the system 102, and the system 102 can process the raw data for use in the operations described herein.

In some embodiments, any of the above data can be queried by one or more of the user devices 104 from one or more databases (e.g., the database 106, a third-party database, etc.). The user device 104 can generate a query and transmit the query to the system 102, which can determine which database may contain requisite information and then connect with that database to execute a query and retrieve appropriate information. In other embodiments, the user device 104 can receive the data directly from the third-party database and transmit the received data to the system 102, or can instruct the third-party database to transmit the data to the system 102. In some embodiments, the system 102 can include various application programming interfaces (APIs) and/or communication interfaces that can allow interfacing between user devices 104, databases, and/or any other components.

Optionally, the system 102 can also obtain any of the above data from various third party sources, e.g., with or without a query initiated by a user device 104. In some embodiments, the system 102 can be communicatively coupled to various public and/or private databases that can store various information, such as census information, health statistics (e.g., appropriately anonymized), demographic information, population information, and/or any other information. For example, the system 102 can obtain information about health metrics such as blood pressure levels and/or forecasts of blood pressure levels of a plurality of users (e.g., without identifying the users) of the system 102, nutrition data relating to such users, exercise data, social network information, and/or any other information and/or any combination thereof, as described in greater detail below. Additionally, the system 102 can also execute a query or other command to obtain data from the user devices 104 and/or access data stored in the database 106. The data can include data related to the particular user and/or a plurality of patients or other users (e.g., historical blood pressure levels, historical blood glucose concentrations, prior predictions and/or analyses of blood pressure levels and/or blood glucose concentrations, health history data, medical condition history data, exercise history data, nutrition data, etc.), as described herein.

The database 106 can be used to store various types of data obtained and/or used by the system 102. For example, any of the above data can be stored in the database 106. The database 106 can also be used to store data generated by the system 102, such as previous predictions or forecasts produced by the system 102. In some embodiments, the database 106 includes data for multiple users, such as at least 50, 100, 200, 500, 1000, 2000, 3000, 4000, 5000, or 10,000 different users. The data can be appropriately anonymized to ensure compliance with various privacy standards. The database 106 can store information in various formats, such as table format, column-row format, key-value format, etc. (e.g., each key can be indicative of various attributes associated with the user and each corresponding value can be indicative of the attribute's value (e.g., measurement, time, etc.)). In some embodiments, the system 102 obtains data from different types of user devices 104 and/or other data sources, some of which may provide data in disparate formats (e.g., based on the particular software and/or hardware used). In such embodiments, the system 102 can convert the data to a standardized format for storage in the database 106 The standardized format can be configured to facilitate searching and/or retrieval of the data for use in the various methods described herein. Optionally, the standardized format can be organized in a manner suitable for use with the machine learning techniques disclosed herein.

In some embodiments, the database 106 stores one or more tables that can be accessed through queries generated by the system 102 and/or the user devices 104. The tables can store different types of information (e.g., one table can store blood pressure data, another table can store blood glucose data, yet another table can store user health data, etc.), where one table can be updated as a result of an update to another table. Alternatively, a single table can store multiple types of information.

For example, Table 1 below illustrates exemplary health and/or behavioral data that may be provided to the system 102 and/or stored in the database 106. The data in Table 1 can be generated by one or more user devices 104, as previously described. For example, the data can be automatically collected from the user (e.g., from a blood pressure measurement device, fitness tracking device, etc.) and/or may be manually entered by the user (e.g., via a computing device such as a mobile device). Each entry in Table 1 is labeled with a user ID, and includes a time stamp indicating when the data was obtained, the type of data, the data value, and corresponding units of measurement.

TABLE 1
Health and/or Behavioral Data

| User ID | Timestamp | Data Type | Value | Units |
|---|---|---|---|---|
| user 1 | 2019 12 18 10:09:01.195 utc | systolic blood pressure | 122 | mm Hg |
| user 1 | 2019 12 18 10:09:01.198 utc | diastolic blood pressure | 79 | mm Hg |
| user 2 | 2019 12 18 10:09:01.287 utc | systolic blood pressure | 138 | mm Hg |
| user 2 | 2019 12 18 10:09:01.289 utc | diastolic blood pressure | 93 | mm Hg |
| user 1 | 2019 12 18 10:09:02.448 utc | blood glucose | 128 | mg/dL |
| user 3 | 2019 12 18 10:09:02.996 utc | activity | 48 | minutes |
| user 4 | 2019 12 18 10:09:04.828 utc | medicine: chlorothiazide | 500 | mg |
| user 2 | 2019 12 18 10:09:05.108 utc | carbohydrates | 27 | g carbohydrate |
| user 1 | 2019 12 18 10:09:05.218 utc | sodium | 800 | mg |
| user 5 | 2019 12 18 10:09:06.395 utc | medicine: insulin | 4 | U |

As another example, Table 2 below illustrates exemplary personal data that may be provided to the system 102 and/or stored in the database 106. The data in Table 2 can be generated by one or more user devices 104, as previously described. Each entry in Table 2 is labeled with a user ID, and includes personal information for that particular user such as the time zone and/or geographic region in which the user is located, the user's age, the user's gender, and the type of the diabetes the user has (if applicable).

TABLE 2
Personal Data

| User ID | Time Zone | Age | Gender | Diabetes Type |
|---|---|---|---|---|
| user 1 | New York | 44 | M | Type 1 |
| user 2 | Mumbai | 38 | F | N/A |
| user 3 | Los Angeles | 63 | N/A | Type 2 |
| user 4 | Lisbon | N/A | F | Type 2 |

In some embodiments, one or more users can access the system 102 via the user devices 104, e.g., to send data to the system 102 (e.g., blood pressure data, health data, other user data), receive data from the system 102 (e.g., a predicted blood pressure or other health metric), and/or other such operations. The users can be individual users (e.g., patients, healthcare professionals, etc.), computing devices, software applications, objects, functions, and/or any other types of users and/or any combination thereof. The user devices 104 can implement a software application (e.g., a mobile application, web application) configured to receive data from the user (e.g., input manually by the user, automatically sensed from the user), and can use the data to initiate processing by the system 102. For example, upon obtaining appropriate data (e.g., blood pressure data, health data, etc. as discussed above), the user device 104 can generate an instruction and/or command to the system 102, e.g., to process the obtained data, store the data in the database 106, extract additional data from one or more databases, and/or perform analysis of the data. The instruction/command can be in a form of a query, a function call, and/or any other type of instruction/command. In some embodiments, the instructions/commands can be provided using a microphone (e.g., a separate microphone or a microphone imbedded in the user device 104), a speaker, a screen (e.g., using a touchscreen, a stylus pen, and/or in any other fashion), a keyboard, a mouse, a camera, a camcorder, a telephone, a smartphone, a tablet computer, a personal computer, a laptop computer, and/or using any other device. The user device 104 can also instruct the system 102 to perform an analysis of data stored in the database 106 and/or input via the user device 104.

In some embodiments, the system 102 processes and uses the data provided by the user to produce automated decision support. As discussed further below, the system 102 can analyze the obtained data, including past data, continuously supplied data, and/or any other data (e.g., using a statistical analysis, machine learning analysis, etc.), and generate a forecast of an expected health metric (e.g., blood pressure level, blood glucose level, etc.) for the user. Optionally, the system 102 can also provide interpretations, recommendations, notifications, and/or other information related to the obtained data and/or the forecasted health metric. The system 102 can perform such analyses at any suitable frequency and/or any suitable number of times (e.g., once, multiple times, on a continuous basis, etc.). For example, when updated data is supplied to the system 102 (e.g., from the user devices 104), the system 102 can reassess and update its previous prediction, if appropriate. In performing its analysis, the system 102 can also generate additional queries to obtain further information (e.g., from the user devices 104, the database 106, or third party sources). In some embodiments, the user device 104 can automatically supply the system 102 with such information. Receipt of updated and/or additional information can automatically trigger the system 102 to execute a process for reanalyzing, reassessing, or otherwise updating previous predictions.

In some embodiments, the system 102 is configured to forecast the user's health metrics using one or more machine learning models. The machine learning models can include supervised learning models, unsupervised learning models, semi-supervised learning models, and/or reinforcement learning models. Examples of machine learning models suitable for use with the present technology include, but are not limited to: regression algorithms (e.g., ordinary least squares regression, linear regression, logistic regression, stepwise regression, multivariate adaptive regression splines, locally estimated scatterplot smoothing), instance-based algorithms (e.g., k-nearest neighbor, learning vector quantization, self-organizing map, locally weighted learning, support vector machines), regularization algorithms (e.g., ridge regression, least absolute shrinkage and selection operator, elastic net, least-angle regression), decision tree algorithms (e.g., classification and regression trees, Iterative Dichotomiser 3 (ID3), C4.5, C5.0, chi-squared automatic interaction detection, decision stump, M5, conditional decision trees), Bayesian algorithms (e.g., naïve Bayes, Gaussian naïve Bayes, multinomial naïve Bayes, averaged one-dependence estimators, Bayesian belief networks, Bayesian networks), clustering algorithms (e.g., k-means, k-medians, expectation maximization, hierarchical clustering), association rule learning algorithms (e.g., apriori algorithm, ECLAT algorithm), artificial neural networks (e.g., perceptron, multilayer perceptrons, back-propagation, stochastic gradient descent, Hopfield networks, radial basis function networks), deep learning algorithms (e.g., convolutional neural networks, recurrent neural networks, long short-term memory networks, stacked auto-encoders, deep Boltzmann machines, deep belief networks), dimensionality reduction algorithms (e.g., principle component analysis, principle component regression, partial least squares regression, Sammon mapping, multidimensional scaling, projection pursuit, discriminant analysis), time series forecasting algorithms (e.g., exponential smoothing, autoregressive models, autoregressive with exogenous input (ARX) models, autoregressive moving average (ARMA) models, autoregressive moving average with exogenous inputs (ARMAX) models, autoregressive integrated moving average (ARIMA) models, autoregressive conditional heteroskedasticity (ARCH) models), and ensemble algorithms (e.g., boosting, bootstrapped aggregation, AdaBoost, blending, stacking, gradient boosting machines, gradient boosted trees, random forest). Additional examples of machine learning models suitable for use with the forecasting techniques herein are discussed further below.

Although FIG. 1 illustrates a single set of user devices 104, it will be appreciated that the system 102 can be operably and communicably coupled to multiple sets of user devices, each set being associated with a particular patient or user. Accordingly, the system 102 can be configured to receive and analyze data from a large number of users (e.g., at least 50, 100, 200, 500, 1000, 2000, 3000, 4000, 5000, or 10,000 different users) over an extended time period (e.g., weeks, months, years). The data from these users can be used to train and/or refine one or more machine learning models implemented by the system 102, as described below.

The system 102 and user devices 104 can be operably and communicatively coupled to each other via the network 108. The network 108 can be or include one or more communications networks, and can include at least one of the following: a wired network, a wireless network, a metropolitan area network (“MAN”), a local area network (“LAN”), a wide area network (“WAN”), a virtual local area network (“VLAN”), an internet, an extranet, an intranet, and/or any other type of network and/or any combination thereof. Additionally, although FIG. 1 illustrates the system 102 as being directly connected to the database 106 without the network 108, in other embodiments the system 102 can be indirectly connected to the database 106 via the network 108. Moreover, in other embodiments one or more of the user devices 104 can be configured to communicate directly with the system 102 and/or database 106, rather than communicating with these components via the network 108.

The various components 102-108 illustrated in FIG. 1 can include any suitable combination of hardware and/or software. In some embodiment, components 102-108 can be disposed on one or more computing devices, such as server(s), database(s), personal computer(s), laptop(s), cellular telephone(s), smartphone(s), tablet computer(s), and/or any other computing devices and/or any combination thereof. In some embodiments, the components 102-108 can be disposed on a single computing device and/or can be part of a single communications network. Alternatively, the components can be located on distinct and separate computing devices.

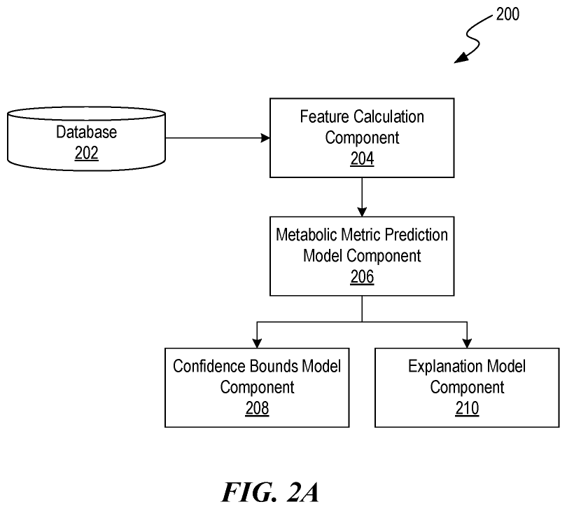

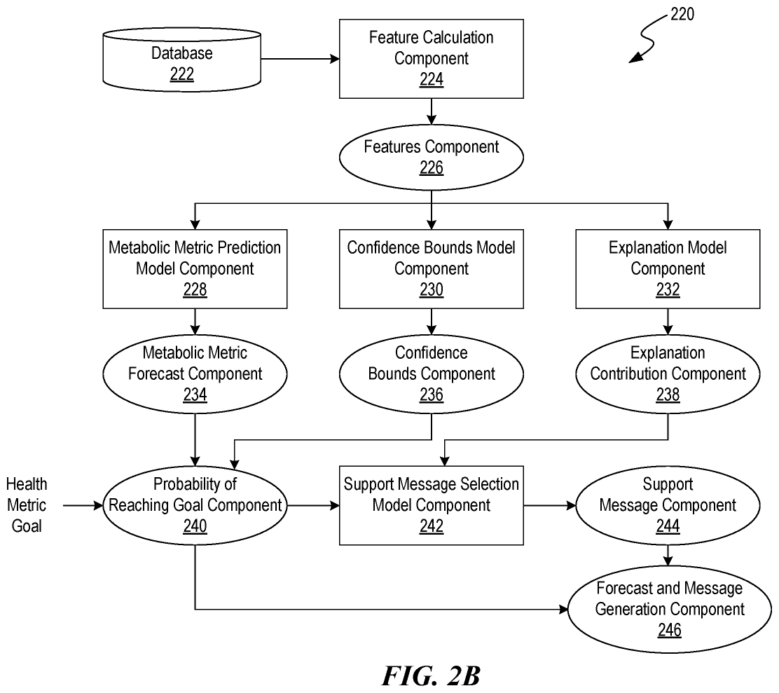

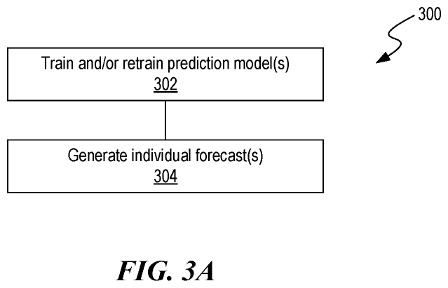

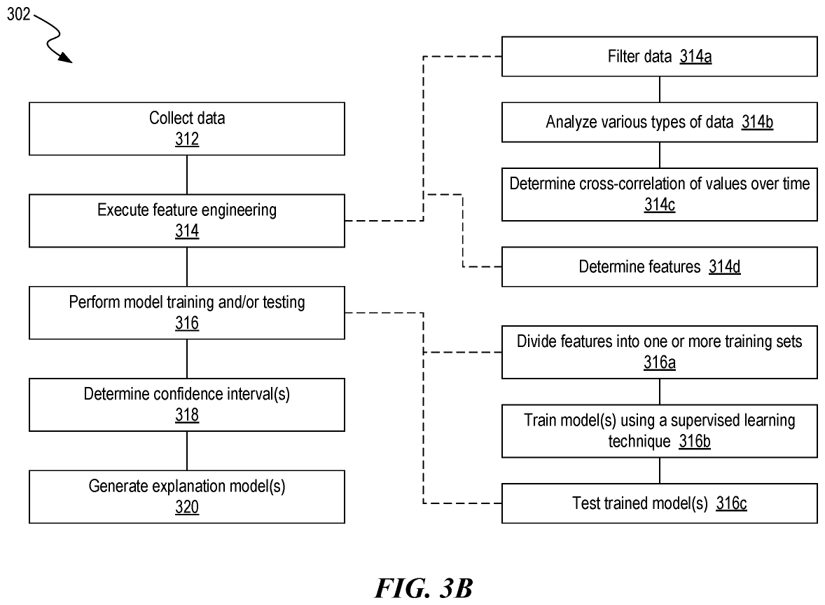

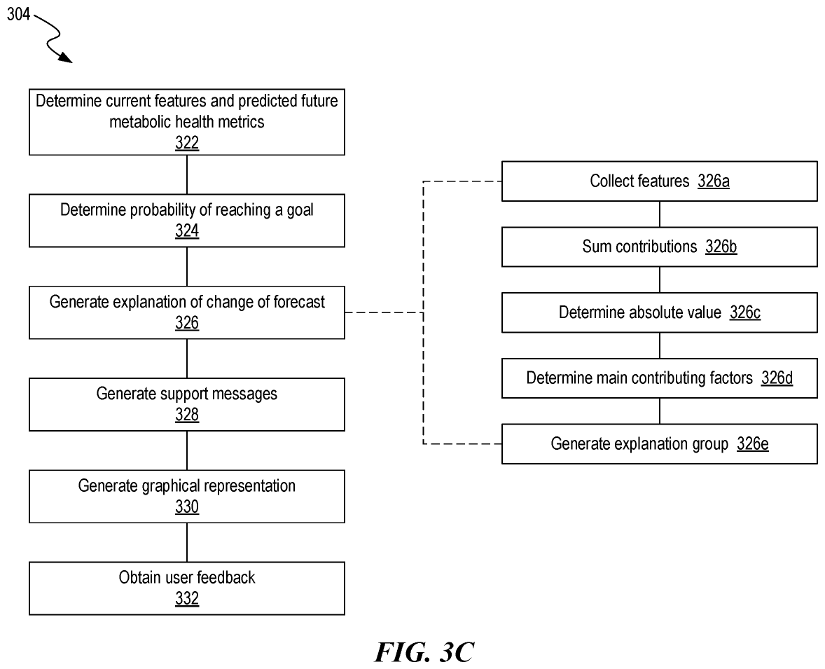

FIG. 2A illustrates a training/retraining system 200 (“system 200”) configured in accordance with embodiments of the present technology. The system 200 can be configured to execute operations associated with training and/or retraining a forecasting machine learning model, as described further below. The system 200 can include a database 202 (which can be similar to the database 106 of FIG. 1), a feature calculation component 204, a metabolic metric (e.g., blood pressure, blood glucose concentration, etc.) prediction model component 206, a confidence bounds model component 208, and an explanation model component 210. In some embodiments, some or all of the components 202-210 may be incorporated into one or more components of the computing environment 100 of FIG. 1, such as the system 102, user devices 104, and/or database 106. Alternatively or in combination, any of the components 202-210 can be disposed on one or more computing devices, such as, server(s), database(s), personal computer(s), laptop(s), cellular telephone(s), smartphone(s), tablet computer(s), and/or any other computing devices and/or any combination thereof. In some embodiments, the components 202-210 may be disposed on a single computing device and/or may be part of a single communications network. Alternatively, the components can be located on distinct and separate computing devices. Functionalities of each of these components are discussed further below in connection with FIGS. 3A-3B.

FIG. 2B illustrates a prediction generation system 220 (“system 220”) configured in accordance with embodiments of the present technology. The system 220 can be configured to execute operations associated with generating a prediction of a metabolic health metric (e.g., blood pressure, blood glucose concentration, weight, etc.), as described further below. The system 220 can include a database 222 (which can be similar to the database 106 of FIG. 1 and/or the database 202 of FIG. 2A), a feature calculation component 224 (which can be similar to the feature calculation component 204 of FIG. 2A, a features component 226, a metabolic metric prediction model component 228 (which can be similar to the metabolic metric prediction model component 206 of FIG. 2A), a confidence bounds model component 230 (which can be similar to the confidence bounds model component 208 of FIG. 2A), an explanation model component 232 (which can be similar to the explanation model component 210 of FIG. 2A), a metabolic metric forecast component 234, a confidence bounds component 236, an explanation contribution component 238, a probability of reaching goal component 240, a support message selection model component 242, a support message component 244, and a forecast and message generation component 246. In some embodiments, some or all of the components 222-246 may be incorporated into one or more components of the computing environment 100 of FIG. 1, such as the system 102, user devices 104, and/or database 106. Alternatively or in combination, any of the components 222-246 can be disposed on one or more computing devices, such as, server(s), database(s), personal computer(s), laptop(s), cellular telephone(s), smartphone(s), tablet computer(s), and/or any other computing devices and/or any combination thereof. In some embodiments, the components 222-246 may be disposed on a single computing device and/or may be part of a single communications network. Alternatively, the components can be located on distinct and separate computing devices. Functionalities of each of these components are discussed below in connection with FIGS. 3A and 3C.

### Methods for Forecasting Health Metrics

FIGS. 3A-3C are flow charts illustrating a method or process 300 for forecasting a health metric of a user, in accordance with embodiments of the present technology. Specifically, FIG. 3A illustrates the overall process 300, FIG. 3B illustrates additional details of the process of block 302 of FIG. 3A, and FIG. 3C illustrates additional details of the process of block 304 of FIG. 3A. The process 300 can be executed by any embodiment of the systems and the devices described herein, such as the system 102 and/or user devices 104 of FIG. 1, the system 200 of FIG. 2A, the system 220 of FIG. 2B, or any suitable combination thereof. The process 300 can be used to generate a forecast of a health metric for a user, as well as provide an interpretation of health data and/or generated forecast for the user. As may be understood, the process 300 may be used for any other forecasting and/or interpretation purposes. For ease of illustration, the following description will refer to forecasting and interpretation of metabolic health metrics.

The process 300 may be executed for any suitable user, including those that may have been diagnosed (or have the same and/or similar and/or related conditions) with hypertension, cardiovascular disorders, diabetes, and/or any other conditions. In other embodiments, however, the process 300 can be executed for a user who has not been diagnosed with a specific condition, e.g., a user who is simply seeking to improve and/or maintain their state of health. Further, the process 300 may be configured to generate metabolic health metric forecasts (e.g., whether the user's blood pressure is likely to increase, decrease, or remain unchanged, etc.) during a predetermined period of time, at a predetermined time point, etc. By way of a non-limiting example, the process 300 may be configured to generate a forecast of metabolic health metric values that may be expected 30 days from the start of the forecast process execution.

Referring first to FIG. 3A, at block 302, the process 300 includes training and/or retraining one or more prediction models for performing forecasting of metabolic health metrics. The prediction models can be or include machine learning models, such as any of the machine learning models described herein. The training and/or retraining process performed in connection with block 302 (“process 302”) can include multiple sub-steps, as shown in FIG. 3B.

Referring next to FIG. 3B, at block 312, the process 302 includes collecting various data. The data can include any of the health and/or user data described herein (e.g., in connection with FIGS. 1-2B), and can include data for a single user and/or multiple users. For example, the data may include data values for and/or related to blood pressure, heart rate, blood glucose, weight, laboratory results (e.g., HbA1c), physical activity, medications, food consumed, sleep, age, basal energy consumption, oxygen consumption, and/or any other suitable data, as previously discussed. For example, the data collected in block 312 can include health and/or behavioral data for a plurality of users, e.g., such as any of the data shown in Table 1 above. As another example, the collected data can include personal data, such as any of the data shown in Table 2 above.

In some embodiments, the input data may include at least one of the following: current and/or previous blood pressure measurement data of a user and/or other users; current and/or previous heart rate measurement data of the user and/or other users; current and/or previous blood glucose measurement data of the user and/or other users; meal characteristics data (e.g., number of meals, time of meals, grams carbohydrates and other nutrients (e.g., salt, cholesterol, etc.) consumed during meal times, whether currently and/or in the past), and/or any other meal data); physical exercise data (e.g., workout times, activity type (e.g., walking, running, etc.); current and/or previous weight, height, BMI, etc. data of the user and/or other users; current and/or previous HbA1c data values of the user and/or other users; medical history data related to the user and/or other users (e.g., family history, user health history, diagnoses, blood pressure, etc.); prescription and non-prescription medication data; sleep data; various optional personal data (e.g., geographic location, ethnicity, race, gender, etc.); diagnostic data, etc. Any of the above data types may be also obtained from other users (which may be anonym ized, if appropriate).

The input data can be obtained in many different ways, such as via manual entry by the user and/or other users, collected automatically through one or more sensors, activity trackers, smart watches, smartphones, medical equipment, and/or any other devices and/or systems. In some embodiments, for example, the data is collected from one or more data sources, such as the database 106 of FIG. 1 and/or the database 202 of FIG. 2A. Optionally, the data can be entered manually, such as via one or more graphical user interfaces (e.g., presented by one or more of the user devices 104 of FIG. 1). Alternatively or in combination, the data can be automatically obtained and/or passively collected using one or more devices, databases, computing systems, storage locations, etc. In some embodiments, the data can be organized and/or otherwise processed into a format suitable for use in training one or more machine learning models. For example, the collected data (e.g., as shown in Tables 1 and 2) may initially be in an irregular and/or non-standardized format, e.g., data entries may be irregularly spaced in time, different users may have different numbers of data entries, the data logging frequency for a user may vary over time, etc. The collected data can be processed for machine learning purposes by calculating one or more features based on the data, and the features can be used as inputs into the machine learning model(s). A representative example of a process for calculating features from input data is described in detail below.

At block 314, the process 302 can include executing a feature engineering process (e.g., using the feature calculation component 204 of FIG. 2A). This process may be executed for each user and/or for a plurality of users. During this process, the collected data may be converted into model features and/or model inputs in accordance with blocks 314a-314d shown in FIG. 3B.

At block 314a, the input data can be filtered using one or more filters. For example, the input data can be filtered to exclude one or more data values that are determined to be invalid, e.g., values that may be too high and/or low to be possible for a particular health measurement (e.g., blood pressure, blood glucose, heart rate, weight, etc.). As another example, one or more outlier values (e.g., values that are many standard deviations away from all prior entries for that user) can also be excluded. In yet another example, duplicate values can also be excluded. Additionally, contradictory values can be removed or excluded from further calculations. For example, if two values of the same health measurement have different values but are separated by a very short time (e.g., less than one minute), either the first, the last, or an average of the two values may be used. The selection of the particular value may be dependent on the specific health measurement.

At block 314b, the process 302 can include analyzing data values relating to various types of health measurements. In some embodiments, for example, block 314b includes calculating averages and/or other statistics values for one or more health measurements, such as blood pressure (e.g., systolic and/or diastolic), sleep, heart rate, blood glucose, doses of medications, carbohydrates and/or other nutrients, physical activity, etc. The analysis may be performed at specific times, e.g., at the times when blood pressure has been measured.

At block 314c, the process 302 can include determining cross-correlations between values. In some embodiments, the cross-correlation may be computed over time between averages of systolic and diastolic blood pressures, blood pressure and heart rate, blood pressure and blood glucose, and/or other pairs and/or groups of variables corresponding to various health measurements.

At block 314d, the process 302 can include determining features for use in metabolic health metric forecasting. This can include computation of features of changes in statistics of historical averages and/or any other determined values. For example, the features can include values (e.g., the most recent value or set of values) and/or statistics (e.g., averages, standard deviations, ranges, sums, differences, ratios, maximums, minimums, percentiles, probabilities, cross-correlations, time-in-range) for any suitable health data, including, but not limited to, any of the following: blood glucose levels, blood pressure levels, heart rate, weight, food intake, medical history, demographics, diagnoses and/or medical conditions, medications, sleep patterns, activity patterns, and/or combinations thereof. Features can be computed across health data obtained over any suitable length of time (e.g., 15 days, 30 days, 60 days, or 90 days) and at any suitable time period before the prediction is made (e.g., immediately before the prediction, 15 days before the prediction, 30 days before the prediction, 60 days before the prediction, or 90 days before the prediction).

Optionally, the features can be categorized into feature groups, also referred to herein as “explanation groups.” Each feature group can be associated with a corresponding health factor, such that features within the same feature group are all related to the same underlying health factor, e.g., all features in a “blood pressure” feature group are computed from the user's blood pressure data, all features in a “blood glucose” feature group are computed from the user's blood glucose data, etc. Examples of health factors that may be used to determine feature groups include, but are not limited to: blood pressure (e.g., blood pressure history), blood glucose (e.g., blood glucose history), heart rate (e.g., heart rate history), multi-factor interactions (e.g., blood pressure-glucose interactions, blood pressure-heart rate interactions), demographic factors, meal intake (e.g., food history), sleep (e.g., sleep history), weight (e.g., weight history), activity (e.g., activity history), medical conditions and/or diagnoses, and the like.

Table 3 below illustrates a representative set of determined features and their corresponding explanation groups, in accordance with embodiments of the present technology.

TABLE 3
Determined Features.

| Feature | Explanation Group |
|---|---|
| a1c | blood glucose history |
| last blood glucose | blood glucose history |
| previous a1c | blood glucose history |
| blood glucose 30 days mean | blood glucose history |
| blood glucose 30 days std | blood glucose history |
| probability of being “low” (hypo) | blood glucose history |
| probability of being high (hyper) | blood glucose history |
| blood glucose 30 days avg range | blood glucose history |
| blood glucose percentiles - 10%, 25%, | blood glucose history |
| 75%, 90% | |
| cross correlation of blood pressure systolic | blood pressure - glucose |
| and blood glucose | interactions |
| cross correlation of blood pressure systolic | blood pressure - heart rate |
| and heart rate | interactions |
| last blood pressure (systolic) | blood pressure history |
| last blood pressure (diastolic) | blood pressure history |
| blood pressure systolic 90 days average | blood pressure history |
| blood pressure systolic 90 days std | blood pressure history |
| blood pressure systolic 30 days average | blood pressure history |
| blood pressure systolic 30 days std | blood pressure history |
| blood pressure diastolic 90 days average | blood pressure history |
| blood pressure diastolic 90 days std | blood pressure history |
| blood pressure diastolic 30 days average | blood pressure history |
| blood pressure diastolic 30 days std | blood pressure history |
| cross correlation of blood pressure systolic | blood pressure history |
| vs diastolic | |
| change in mean systolic blood pressure | blood pressure history |
| from N days ago | |
| change in mean diastolic blood pressure | blood pressure history |
| from N days ago | |
| gender | demographic factors |
| location | demographic factors |
| age | demographic factors |
| daily carbs amount - mean of 30 days | food history |
| heart rate 90 days average | heart rate history |
| heart rate 90 days std | heart rate history |
| heart rate 30 days average | heart rate history |
| heart rate 30 days std | heart rate history |
| diabetes type | medical conditions |
| daily insulin amount - mean of 30 days | medications |
| sleep duration 90 days average | sleep history |
| sleep duration 90 days std | sleep history |
| sleep duration 30 days average | sleep history |
| sleep duration 30 days std | sleep history |
| previous weight | weight |
| weight | weight |

At block 316, the process 302 can include performing model training and/or testing (e.g., using the metabolic metric prediction model component 206 of FIG. 2A). In some embodiments, the model is trained to predict a value and/or range for a health metric at a predetermined future time period. For example, the model(s) can be configured to predict a 30-day average blood pressure (e.g., systolic and/or diastolic blood pressure) at 1 month, 2 months, 3 months, 4 months, 5 months, 6 months, 7 months, 8 months, 9 months, 10 months, 11 months, 12 months, or more, in the future. The training and/or testing of the model may be executed based on inputs in accordance with blocks 316a-316c of FIG. 3B.

At block 316a, the process 302 can include dividing the determined features into one or more training sets and/or one or more test sets. For example, a training set can include all features and/or records prior to a cut-off date, and a test set can include all feature and/or records after the cut-off date. As can be understood, any desired training and/or test sets can be generated.

In some embodiments, features are calculated at each time new data becomes available. However, the features used for purposes of training and/or testing can be determined using data points for a specific user that are sufficiently far apart in time. Features and/or targets calculated at nearby times may be based on many of the same data points, which may be undesirable for prediction accuracy. For example, a 30-day average blood pressure ending on April 20th may use many of the same blood pressure measurements as a 30-day average blood pressure ending on April 21st. The use of such nearby points in the training and/or testing sets may bias the resulting model in undesirable ways that may overstate and/or degrade the model's predictive power. To reduce or prevent the occurrence of these issues, the process 302 can set a minimum time between data points for training and/or testing purposes for each user.

Table 4 illustrates representative minimum separation time values and the corresponding improvements in model accuracy, in accordance with embodiments of the present technology. Although the data in Table 4 is shown for blood pressure predictions, it will be appreciated the approaches described herein can be applied to predictions of other types of health metrics (e.g., blood glucose, weight, etc.). Table 4 shows improvements in model accuracy calculated across all users and for users having a 30-day average blood pressure value greater than 135 mmHg at the time of the forecast. In Example 1 of Table 4, the minimum separation time between data points was selected based on the desired forecast horizon to meet a requirement that adjacent data points are based on time periods that have no more than 3 days' data in common. In Example 2 of Table 4, the minimum data separation was set to 30 days regardless of the forecast horizon. As shown in Table 4, the Example 2 approach provided improvement over the Example 1 approach for shorter forecast horizons. In other embodiments, the separation between data points for training can be selected to improve accuracy. For example, in the cases shown in Table 4, the separation can be specified to be 30 days, or 30 days less than the forecast horizon, whichever is greater.

TABLE 4
Minimum separation time values.

| | | | |
|---|---|---|---|
| | Minimum | Accuracy | Accuracy |
| Forecast | Data | Improvement, | Improvement, Blood |
| Horizon | Separation | All Users | Pressure > 135 mmHg |

Example 1

| | | | |
|---|---|---|---|
| 1-2 months | 20 days | 14.0% | 14.6% |
| 2-3 months | 45 days | 14.6% | 16.4% |
| 3-4 months | 60 days | 17.3% | 18.0% |
| 4-6 months | 90 days | 19.1% | 22.3% |

Example 2

| | | | |
|---|---|---|---|
| 1-2 months | 30 days | 16.5% | 20.0% |
| 2-3 months | 30 days | 15.8% | 19.1% |
| 3-4 months | 30 days | 15.1% | 18.1% |
| 4-6 months | 30 days | 14.3% | 13.3% |

At block 316b, the process 302 can include training the model(s) using a supervised learning model technique. The model(s) can include, for example, a Gradient Boosted Trees model and/or any other suitable machine learning model configured to learn a mapping from a set of input points to a desired target value. The model(s) can be trained on data from a plurality of users simultaneously, so that experiences of one or more users may be used to inform predictions of other users having similar features. This can allow the model(s) to make predictions even for users with few data points and/or with irregular and/or incomplete input data.

At block 316c, the process 302 can include testing the trained model(s). For example, the trained model(s) can be used to predict metabolic health metric values in a test set. The predicted metrics can then be compared to the actual metrics to determine the accuracy of the model(s). If the model accuracy is not satisfactory, the model(s) can be re-trained, e.g., using additional and/or different training sets. Optionally, different candidate feature sets can be compared (e.g., based on accuracy of the resulting training model(s)), and the feature set that generated the best testing performance can be selected for future use.

One of skill in the art will appreciate that there are many measures of accuracy. For example, one measure can include a comparison of the model skill with a naïve predictor, such as persistence (e.g., “predict that the future value will be the same as the current value”). The model score can be generated and can correspond to a relative reduction in root mean square error (RMSE) compared to the naïve predictor, e.g., 1−RMSE_{model}/RMSE_{naïve}. This measure will equal 1 if the model prediction has zero error, and will equal zero if the model prediction has the same error as the naïve prediction. If the model errors are larger than the naïve errors, this accuracy measure will be negative.

At block 318, the process 302 can optionally include determining one or more confidence intervals for the forecasted data (e.g., using the confidence bounds model component 208 of FIG. 2A). The confidence intervals can be determined in many different ways. For example, in some embodiments, the training models used can include an XGBoost model or similar model including a large number of “trees” (e.g., at least 150 trees). For ease of illustration only, the following description will refer to the tree-based models identified above. However, in other embodiments, the process 300 can use any suitable training model(s) and the present technology is not limited to the XGBoost or similar such models (e.g., Gradient Boosted Trees models, etc.).

In such models, each data item may correspond to one “leaf” of each tree, and each leaf may have a “weight” that can be determined when the model is trained. The predicted value for that data item may be the sum of the weights of the leaves that that data item ends up in for each tree. The weights may be determined as follows: each tree's contribution may be considered to be a correction to the running sum. For example, to compute weights in a leaf of a 4th tree, the training routine can look at the predictions for the items in that leaf from summing their weights from the first three trees. All those 3-tree predictions may have some error, and the mean value of that 3-tree error for those items becomes the correction value, or the weight of the tree-4 leaf being computed. As such, the weight of that tree-4 leaf may be the mean value of the 3-tree errors of the items in that leaf multiplied by a value that depends on the model parameters being trained.

In some embodiments, the weight does not consider the spread of the 3-tree errors. For example, the errors of items in the tree-4 leaf could range from 10 to 12, with an average value of 11, or they could range from −89 to +202, with an average value of 11: the weight may be the same. However, the 4-tree prediction errors may be larger in the second case than in the first. In some embodiments, the process 302 can assume that if the prediction is the sum of each leaf's errors' mean, then the variance of the prediction may be the sum of each leaf's errors' variance. The process 302 can then examine the trained model (e.g., XGBoost model) and determine the variance of errors in each leaf of each tree, and turn that into a “variance model.” The inputs to the variance model may be the same as the inputs to the prediction model: for each prediction, the variance model adds up the variances of the leaf that prediction lands in for each tree.

The prediction errors may not be normally distributed; however, the error distributions may be very close between training and test sets. Next, the error distribution for training set predictions with the same variance as the prediction in question may be ascertained. The quantiles of that training set distribution may then be assumed to be the quantiles of the prediction in question.

At block 320, the process 302 can optionally include generating one or more explanation models (e.g., using the explanation model component 210 of FIG. 2A). The explanation model(s) may be generated based on the trained model(s) using any suitable attribution algorithm (e.g., Shapley Additive Explanations (SNAP) attribution algorithm, etc.). The inputs to the explanation model may be the inputs used for generation of the forecast, and the explanation model can be configured to determine a contribution (e.g., a marginal contribution) of each input feature to the final value of the forecast. The explanation model may be used to generate an explanation of any particular forecast. In some embodiments, the trained model, associated feature definitions, confidence bound model, and explanation model may then be used to generate metabolic health metric forecasts for one or more users. Further, the generated trained model(s) may be periodically retrained as additional data becomes available, and new predictive features can be tested and/or selected.

Referring back to FIG. 3A, once the appropriate models have been trained and/or retrained, at block 304, the process 300 can be configured to generate individual metabolic health metric forecasts. In some embodiments, generation of individual forecasts can be triggered automatically (e.g., when obtaining new data from one or more devices, database, user inputs), manually (e.g., by a user action, for example, when a blood pressure forecast or any other forecast screen is accessed, the user provides new data, etc.), and/or suitable combinations thereof. The forecast generation process performed in connection with block 304 (“process 304”) can include multiple sub-steps, as shown in FIG. 3C.

Referring next to FIG. 3C, upon triggering of the forecast, at block 322, the process 304 includes determining current features and predicted future metabolic health metrics. The determination can be performed using the database 222 and/or components 224-232 of FIG. 2B. In some embodiments, for example, when a forecast is triggered, a request may be transmitted from a user device 104 to the system 102 of FIG. 1A (e.g., a server), where the user's data can be accessed, and features can be determined. The features may then be transmitted to a forecasting and/or analysis engine for executing the generated model(s), which may be used to forecast a value of a target metabolic health metric (e.g., a target blood pressure, etc.).

At block 324, the process 304 can include determining a probability of the user reaching a health metric goal (e.g., using the probability of reaching goal component 240 of FIG. 2B). For example, the model(s) described herein can be configured to forecast a probability that the user's metabolic health metric will be higher (or lower) in n number of months, weeks, days, and/or any other predetermined period of time. In some embodiments, to determine a probability of reaching a particular goal, the input parameters and/or data used for the metabolic health metric model may be used in the confidence bounds model to generate quantiles of likely future metabolic health metric, e.g., 10% probability that blood pressure will be less than 110 mmHg, 25% probability that blood pressure will be less than 150 mmHg, etc. As can be understood, each user may have a specific clinical goal, e.g., a mean systolic blood pressure less than 130 mmHg. The confidence bounds model may be used to determine a probability that future blood pressure will be lower than the goal value, e.g., “38% probability that blood pressure will be less than 130 mmHg,” meaning there is 38% probability that the user will meet their goal in the time horizon of the forecast.

At block 326, the process 304 can include generating an explanation of change of forecast (e.g., using the components 234-238 of FIG. 2B). The explanation can indicate to the user which health factors (e.g., blood pressure, blood glucose, weight, etc.) were most significant in influencing the forecast outcome. In some embodiments, the explanation links the forecast outcome to groups of features (e.g., explanation groups), rather than to individual features. This approach may be advantageous because the meaning of a specific feature may be opaque or confusing to the user (e.g., “30 day standard deviation of systolic blood pressure values”), whereas the meaning of a larger explanation group may be easier for the user to understand (e.g., “blood pressure”). Accordingly, the approach described herein can help the user understand the forecast, and also give clear and actionable guidance for how to achieve the user's health goals.

The forecast explanation can be generated in many different ways. For example, as in the previous step (block 324), the same features used as inputs to the forecast model(s) can also be used as inputs to the explanation model(s). The explanation model(s) can be configured to determine a contribution of each feature to the forecast value. These contributions may be positive (e.g., factors increasing the likelihood of reaching a specific blood pressure goal, weight goal, etc.) and/or negative (e.g., decreasing the likelihood of reaching a specific blood pressure goal, weight goal, etc.). In some embodiments, certain features and/or feature groups may be more influential and/or contribute more to the forecast than other features and/or feature groups (e.g., meal intake, weight, blood glucose concentration, age, etc.). As such, the forecast explanation may make a reference to those features and/or feature groups directly, e.g., the user is likely/unlikely to reach a specific blood pressure goal “based on your salt intake and heart rate history.” In some embodiments, many data contributions, corresponding to respective features and/or feature groups, can be configured to affect the final forecast determination, as illustrated by blocks 326a-326e shown in FIG. 3C.

At block 326a, the process 304 can include collecting one or more features that can be used in generation of a forecast. As previously discussed, the features can include values and/or statistics for one or more of the following data points: blood glucose levels, blood pressure levels, heart rate, weight, food intake, medical history, demographics, diagnoses and/or medical conditions, medications, sleep patterns, activity patterns, and/or combinations thereof. One or more obtained features can be grouped in one or more feature or explanation groups, with each explanation group being associated with a corresponding health factor (e.g., blood pressure, blood glucose, heart rate, demographics, diagnoses, meal intake, sleep, weight, activity, etc.). Representative examples of features and explanation groups are illustrated in Table 3 above, and also described further below with reference to Tables 5A-6B.

At block 326b, for each forecast, the process 304 can include summing the contributions of each feature within each explanation group into subtotals, to determine the individual contribution of each explanation group to the forecast. The subtotal for an explanation group may be positive, negative, or zero. The overall forecast value may then be equal to the sum of each group's subtotal. As previously discussed, the contributions of each feature can be determined using any suitable technique, such as a SHAP attribution algorithm or other attribution algorithm.

At block 326c, the process 304 can include determining the absolute values of each explanation group's subtotal to determine the corresponding magnitudes of each subtotal. The magnitude of the subtotal may be proportional to the contribution of that explanation group to the forecast, e.g., a larger magnitude indicates a larger contribution, a smaller magnitude indicates a smaller contribution, a zero magnitude indicates no contribution.

At block 326d, the process 304 can include determining the main contributing factors (e.g., weight, blood glucose, meal intake, etc.) to the forecast. The main contributing factors may be determined by selecting the explanation groups having the largest magnitudes. Any number of explanation groups may be selected, e.g., a fixed number (e.g., the top three groups), a number that depends on their contribution values (e.g., define the gross total change as the sum of the magnitudes, and select a sufficient number of groups to collectively account for at least 95% of the gross total change (or any other suitable percentage)), etc.

At 326e, the process 304 can generate explanation groups that are likely to be the driving explanatory factors for the forecast (e.g., “based mainly on your sleeping and eating patterns”). In some embodiments, in order to understand factors leading to the change in, for example, a blood pressure forecast from a prior blood pressure forecast, explanatory contributions of the two forecasts may be compared. The difference in contributions may explain the change in likelihood of success of achieving a health metric goal. For example, if the forecast probability of success has increased by five percentage points, and all contributions are the same in both forecasts except for diet (e.g., which has increased by five percentage points), then the improvement in the forecast may be due to improvements in the diet. In some embodiments, changes in contributions from one forecast to the next forecast may be aggregated and/or filtered using processes described above to generate a “digest” of main factors contributing to the change in forecasted blood pressure values.

Tables 5A-6B illustrate representative examples of experimental determinations of feature group contributions for generating an explanation of a forecast or prediction, in accordance with embodiments of the present technology. Although Tables 5A-6B are described with reference to a prediction of a blood pressure health metric, it will be appreciated that similar tables and/or calculations may be generated for any other suitable health metric. The following abbreviations are used in Tables 5A-6B: “bp”—blood pressure, “bg”—blood glucose concentration, “hr”—heart rate, “demo/diag”—demographics/diagnosis, “med”—medications taken, “food”—meals ingested, “weight”—weight measurement, “activity”—exercise data.

Table 5A lists each feature used in the prediction, a description of the feature, the feature or explanation group to which each feature belongs, the SHAP-value contribution of each feature to the three-month and one-month predictions, and the change of SHAP value between the 3 month and 1 month predictions for each feature. The “Value 3 Months Prediction” and “Value 1 Month Prediction” columns represent deviations from a baseline blood pressure value (in this experimental example, 129.9 mmHg) for predictions made three months and one month before the target date, respectively. As can be seen in Table 5A, the total deviation summed across all features for the three-month prediction is approximately −4.0, and the total deviation for the one-month prediction is approximately −7.4.

TABLE 5A
Feature Grouping, Prediction Values, and Changes in Prediction

| | | | Value 3 | Value 1 | Change |
|---|---|---|---|---|---|
| | | Feature | Months | Month | Since Last |
| Feature Name | Feature Description | Group | Prediction | Prediction | Prediction |
| blood_pressure | last systolic blood | bp | −0.144 | −0.064 | 0.080 |
| _sys | pressure | | | | |
| weight | last entered weight | weight | 0.167 | 0.068 | −0.099 |
| blood_pressure | last diastolic blood | bp | 0.009 | 0.004 | −0.005 |
| _sto | pressure | | | | |
| weight_prev | difference between two | weight | 0.150 | 0.007 | −0.144 |
| | last weights recorded | | | | |
| blood_glucose_ | blood glucose mean since | bg | −0.235 | 0.006 | 0.241 |
| mean | inception | | | | |
| blood_glucose_ | blood glucose standard | bg | −0.009 | 0.001 | 0.010 |
| std | deviation since inception | | | | |
| low_prob | probability of blood | bg | −0.016 | 0.001 | 0.017 |
| | glucose getting below 70 | | | | |
| | mldL | | | | |
| high_prob | probability of blood | bg | 0.007 | −0.012 | −0.019 |
| | glucose getting above 180 | | | | |
| | mldL | | | | |
| blood_pressure | systolic blood pressure 90 | bp | −2.537 | −4.218 | −1.681 |
| _sys_mean_90 | days average | | | | |
| blood_pressure | systolic blood pressure 90 | bp | 0.322 | −0.209 | −0.531 |
| _sys_std_90 | days standard deviation | | | | |
| blood_pressure | systolic blood pressure 30 | bp | −0.758 | −1.683 | −0.925 |
| _sys_mean_30 | days average | | | | |
| blood_pressure | systolic blood pressure 90 | bp | −0.107 | −0.053 | 0.054 |
| _sys_std_30 | days standard deviation | | | | |
| insulin_30d_su | sum of insulin unit taken in | med | −0.006 | −0.003 | 0.003 |
| m | 30 days | | | | |
| carbs_30d_su | sum of carb units | food | −0.078 | −0.048 | 0.030 |
| m | taken/reported in 30 days | | | | |
| blood_glucose_ | average over 30 days of | bg | −0.003 | 0.000 | 0.002 |
| 30d_avg_range | blood glucose daily range | | | | |
| bp_crcorr | pearson correlation | bp | −0.157 | 0.070 | 0.227 |
| | coefficient of 30 days | | | | |
| | average systolic and | | | | |
| | diastolic blood pressure | | | | |
| last_0_blood_p | last systolic BP 30 days | bp | −0.246 | −0.581 | −0.438 |
| ressure_sys_m | average | | | | |
| ean_30 | | | | | |
| diff_1_blood_pr | change over systolic BP | bp | 0.024 | −0.220 | −0.244 |
| essure_sys_me | 30 days average from | | | | |
| an_30 | previous measurement | | | | |
| diff_6_blood_pr | change over systolic BP | bp | 0.060 | −0.158 | −0.218 |
| essure_sys_me | 30 days average from 6 | | | | |
| an_30 | measurements before last | | | | |
| diff_12_blood_ | change over systolic BP | bp | −0.012 | 0.020 | 0.032 |
| pressure_sys_ | 30 days average from 12 | | | | |
| mean_30 | measurements before last | | | | |
| diff_24_blood_ | change over systolic BP | bp | −0.431 | 0.276 | 0.707 |
| pressure_sys_ | 30 days average from 24 | | | | |
| mean_30 | measurements before last | | | | |
| mean_30D_tar | systolic BP 30 days mean | bp | −0.092 | −0.215 | −0.224 |
| get | | | | | |
| min_30D_targe | systolic BP 30 days | bp | 0.027 | 0.008 | −0.019 |
| t | minimum | | | | |
| max_30D_targ | systolic BP 30 days | bp | 0.004 | −0.081 | −0.084 |
| et | maximum | | | | |
| med_30D_targ | systolic BP 30 days | bp | 0.149 | 0.030 | −0.210 |
| et | median | | | | |
| std_30D_target | systolic BP 30 days | bp | 0.066 | −0.050 | −0.116 |
| | standard deviation | | | | |
| mean_60D_tar | systolic BP 60 days mean | bp | −0.444 | −0.006 | 0.438 |
| get | | | | | |
| min_60D_targe | systolic BP 60 days | bp | 0.328 | 0.118 | −0.210 |
| t | minimum | | | | |
| max_60D_targ | systolic BP 60 days | bp | −0.040 | −0.075 | −0.035 |
| et | maximum | | | | |
| med_60D_targ | systolic BP 60 days | bp | −0.070 | −0.039 | 0.031 |
| et | median | | | | |
| std_60D_target | systolic BP 60 days | bp | 0.171 | 0.001 | −0.170 |
| | standard deviation | | | | |
| last_activity_30 | 30 days sum of activity | activity | 0.028 | 0.004 | −0.024 |
| d_sum_15D | units 15 days before | | | | |
| | prediction | | | | |
| last_activity_30 | 30 days sum of activity | activity | −0.001 | 0.000 | 0.001 |
| d_sum_30D | units 30 days before | | | | |
| | prediction | | | | |
| last_activity_30 | 30 days sum of activity | activity | 0.000 | 0.000 | 0.000 |
| d_sum_60D | units 60 days before | | | | |
| | prediction | | | | |
| last_heart_rate | 30 days average heart | hr | 0.002 | 0.007 | 0.005 |
| _mean_30_15 | rate 15 days before | | | | |
| D | prediction | | | | |
| last_heart_rate | 30 days average heart | hr | 0.002 | 0.000 | −0.002 |
| _mean_30_30 | rate 30 days before | | | | |
| D | prediction | | | | |
| last_heart_rate | 30 days average heart | hr | −0.002 | −0.001 | 0.001 |
| _ mean_30_60 | rate 60 days before | | | | |
| D | prediction | | | | |
| last_blood_gluc | average over 30 days of | bg | −0.077 | −0.002 | 0.075 |
| ose_30d_avg_r | blood glucose daily range | | | | |
| ange_15D | 15 days before prediction | | | | |
| last_blood_gluc | average over 30 days of | bg | −0.021 | −0.002 | 0.019 |
| ose_30d_avg_r | blood glucose daily range | | | | |
| ange_30D | 30 days before prediction | | | | |
| max_blood_glu | average over 30 days of | bg | −0.018 | −0.005 | 0.013 |
| cose_30d_avg | blood glucose daily range | | | | |
| _range_30D | 60 days before prediction | | | | |
| last_blood_gluc | Blood glucose average 15 | bg | −0.026 | 0.000 | 0.026 |
| ose_30d_avg_r | days before prediction | | | | |
| ange_60D | | | | | |
| max_blood_glu | maximum blood glucose | bg | −0.155 | −0.001 | 0.154 |
| cose_30d_avg | average over last 15 days | | | | |
| _range_60D | before prediction | | | | |
| last_blood_gluc | Blood glucose average 30 | bg | −0.013 | 0.002 | 0.015 |
| ose_mean_30 | days before prediction | | | | |
| D | | | | | |
| max_blood_glu | maximum blood glucose | bg | 0.017 | 0.006 | −0.011 |
| cose_mean_30 | average over last 30 days | | | | |
| D | before prediction | | | | |
| last_blood_gluc | Blood glucose average 60 | bg | −0.008 | 0.000 | 0.008 |
| ose_mean_60 | days before prediction | | | | |
| D | | | | | |
| max_blood_glu | maximum blood glucose | bg | −0.010 | −0.001 | 0.009 |
| cose_mean_60 | average over last 60 days | | | | |
| D | before prediction | | | | |
| last_blood_gluc | blood glucose standard | bg | −0.005 | −0.015 | −0.009 |
| ose_std_15D | deviation 15 days before | | | | |
| | prediction | | | | |
| max_blood_glu | maximum blood glucose | bg | −0.004 | 0.000 | 0.004 |
| cose_std_15D | standard deviation over | | | | |
| | last 15 days before | | | | |
| | prediction | | | | |
| last_blood_gluc | blood glucose standard | bg | −0.006 | 0.000 | 0.006 |
| ose_std_30D | deviation 30 days before | | | | |
| | prediction | | | | |
| max_blood_glu | maximum blood glucose | bg | −0.005 | −0.035 | −0.030 |
| cose_std_30D | standard deviation over | | | | |
| | last 30 days before | | | | |
| | prediction | | | | |
| last_blood_gluc | blood glucose standard | bg | 0.000 | 0.000 | 0.000 |
| ose_std_60D | deviation 60 days before | | | | |
| | prediction | | | | |
| max_blood_glu | maximum blood glucose | bg | 0.009 | 0.020 | 0.011 |
| cose_std_60D | standard deviation over | | | | |
| | last 60 days before | | | | |
| | prediction | | | | |
| last_blood_pre | systolic BP 30 days | bp | −0.183 | −0.069 | 0.114 |
| ssure_sys_mea | average 15 days before | | | | |
| n_30_15D | prediction | | | | |
| max_blood_pre | maximum systolic BP 30 | bp | −0.085 | −0.081 | 0.004 |
| ssure_sys_mea | days average over 15 | | | | |
| n_30_15D | days before prediction | | | | |
| last_blood_pre | systolic BP 30 days | bp | 0.048 | −0.224 | −0.171 |
| ssure_sys_mea | average 30 days before | | | | |
| n_30_30D | prediction | | | | |
| max_blood_pre | maximum systolic BP 30 | bp | −0.039 | −0.022 | 0.017 |
| ssure_sys_mea | days average over 30 | | | | |
| n_30_30D | days before prediction | | | | |
| last_blood_pre | systolic BP 30 days | bp | −0.001 | 0.000 | 0.001 |
| ssure_sys_mea | average 60 days before | | | | |
| n_30_60D | prediction | | | | |
| max_blood_pre | maximum systolic BP 30 | bp | −0.025 | −0.024 | 0.001 |
| ssure_sys_mea | days average over 60 | | | | |
| n_30_60D | days before prediction | | | | |
| last_blood_pre | systolic BP 30 days | bp | −0.008 | −0.010 | −0.002 |
| ssure_sto_mea | standard deviation 15 | | | | |
| n_30_15D | days before prediction | | | | |
| max_blood_pre | maximum systolic BP 30 | bp | −0.009 | 0.000 | 0.009 |
| ssure_sto_mea | days standard deviation | | | | |
| n_30_15D | over 15 days before | | | | |
| | prediction | | | | |
| last_blood_pre | systolic BP 30 days | bp | −0.003 | −0.003 | 0.000 |
| ssure_sto_mea | standard deviation 30 | | | | |
| n_30_30D | days before prediction | | | | |
| max_blood_pre | maximum systolic BP 30 | bp | 0.149 | −0.006 | −0.155 |
| ssure_sto_mea | days standard deviation | | | | |
| n_30_30D | over 30 days before | | | | |
| | prediction | | | | |
| last_blood_pre | systolic BP 30 days | bp | −0.003 | 0.001 | 0.004 |
| ssure_sto_mea | standard deviation 60 | | | | |
| n_30_60D | days before prediction | | | | |
| max_blood_pre | maximum systolic BP 30 | bp | 0.202 | 0.010 | −0.192 |
| ssure_sto_mea | days standard deviation | | | | |
| n_30_60D | over 60 days before | | | | |
| | prediction | | | | |
| gender | gender (0−unknown, 1− | demo/ | 0.016 | 0.000 | −0.016 |
| | male, 2−female) | diag | | | |
| diabetes_type | diabetes type, in case | demo/ | 0.053 | 0.089 | 0.036 |
| | user has diabetes | diag | | | |
| a1c | 1813 last a1c test result | bg | 0.000 | 0.000 | 0.000 |
| | recorded | | | | |
| a1c_prev | difference between two | bg | 0.000 | 0.000 | 0.000 |
| | last a1c tests recorded | | | | |
| heart_rate_me | heart rate 90 days | hr | 0.001 | 0.010 | 0.008 |
| an_90 | average | | | | |
| heart_rate_std_ | heart rate 90 days | hr | −0.003 | −0.002 | 0.001 |
| 90 | standard deviation | | | | |
| heart_rate_me | heart rate 30 days | hr | 0.002 | −0.001 | −0.002 |
| an_30 | average | | | | |
| heart_rate_std_ | heart rate 30 days | hr | −0.015 | 0.000 | 0.014 |
| 30 | standard deviation | | | | |
| sleep_mean_9 | 90 days average of | sleep | −0.001 | 0.000 | 0.001 |
| 0 | number of sleep hours a | | | | |
| | night | | | | |
| sleep_std_90 | 90 days standard | sleep | −0.010 | 0.000 | 0.010 |
| | deviation of number of | | | | |
| | sleep hours a night | | | | |
| sleep_mean_3 | 30 days average of | sleep | 0.000 | 0.000 | 0.000 |
| 0 | number of sleep hours a | | | | |
| | night | | | | |
| sleep_std_30 | 30 days standard | sleep | 0.000 | 0.000 | 0.000 |
| | deviation of number of | | | | |
| | sleep hours a night | | | | |
| | | totals: | −4.004 | −7.359 | |
| | | | | total | −3.355 |
| | | | | change | |
| | | | | since last | |
| | | | | prediction: | |

Table 5B summarizes the prediction data from Table 5A. For the three-month prediction (prediction made July 8 for an October 8 target date), the predicted 30-day average blood pressure value is 129.9 mmHg-4.0 mmHg=125.9 mmHg. For the one-month prediction (prediction made September 9 for an October 8 target date), the predicted 30-day average blood pressure value is 129.9 mmHg-7.4 mmHg=122.5 mmHg. The change between predictions is thus −3.355 mmHg.

TABLE 5B
3 Month and 1 Month Prediction Summary

| Prediction Made: | Jul. 8 | Sep. 9 | |
|---|---|---|---|
| Prediction For: | Oct. 8 | Oct. 8 | Change Since Last Prediction: |
| Predicted Value: | 125.925 | 122.570 | −3.355 |

Table 6A shows feature group subtotals for the change between predictions and Table 6B shows the contribution of each feature group to the change between predictions. The data listed in Table 6A is obtained by summing the “Change Since Last Prediction” data from Table 5A for each feature group to generate each feature group subtotal. In Table 6B, the “Change” column corresponds to the subtotal data from Table 6A, the “Abs (Change)” column lists the magnitude (absolute value) of the subtotal, the “% Of Abs Change” column lists the percentage of the total change in the prediction contributed by each feature group, and the “Cumulative % Of Abs Change” lists the running sum of feature group contributions. As can be seen in Tables 6A and 6B, the top contributing feature groups (e.g., based on absolute values) can account for more than 95% of the total change between predictions. In this example, the main contributors are blood pressure (81% contribution), blood glucose (12% contribution), and weight values (5% contribution). However, as can be understood, in other embodiments, the main contributors may be other factors and/or combinations of factors, such as blood pressure and sleep, blood pressure, food, medications and weight, etc.

TABLE 6A
Feature Group Subtotals

| | Sum of Change |
|---|---|
| | Since Last |
| Feature Group | Prediction: |
| activity | −0.0230356 |
| bg | 0.540250877 |
| bp | −3.71997862 |
| demo/diag | 0.035950523 |
| demo/diag | −0.015542527 |
| food | 0.030264263 |
| hr | 0.025853056 |
| med | 0.00299577 |
| sleep | 0.012269677 |
| weight | −0.243128455 |
| Grand Total | −3.355222036 |

TABLE 6B
Feature Group Contributions

| | | | % Of | Cumulative |
|---|---|---|---|---|
| | | Abs | Abs | % Of Abs |
| Feature Group | Change | (Change) | Change | Change |
| bp (blood pressure) | −3.72 | 3.72 | 81% | 81% |
| bg (blood glucose) | 0.54 | 0.54 | 12% | 92% |
| weight | −0.24 | 0.24 | 5% | 98% |
| food | 0.03 | 0.03 | 1% | 98% |
| activity | −0.02 | 0.02 | 0% | 99% |
| hr (heart rate) | 0.02 | 0.02 | 0% | 99% |
| diagnosis/personal | 0.02 | 0.02 | 0% | 100% |
| sleep | 0.01 | 0.01 | 0% | 100% |
| med | 0.00 | 0.00 | 0% | 100% |
| | Total abs | 4.61 | | |
| | change: | | | |

Referring again to FIG. 3C, once explanation groups are generated, at block 328, the process 304 can optionally generate appropriate support messages (e.g., using components 242-244 of FIG. 2B). For example, an educational and/or encouraging message may be selected based on the generated forecast and explanations. If sleep patterns are reducing the likelihood of success, for example, a message about the importance of sleep and links to further information may be displayed on a user interface.

At block 330, the process 304 can include generating a graphical representation of the results of the process 304 (e.g., using the message generation component 246 of FIG. 2B). For example, the user may be presented a probability of success for meeting a health metrics goal, an explanation of the forecast, a support message appropriate to the probability and the explanation, and/or any other suitable information. In some embodiments, the data may be presented in the following format (that may include, text, images, video, audio, etc.): “Great news! Probability of hitting your goal by January has increased to 78%, because of your great sleep and lower salt! Keep up the amazing work!” The graphical representation may be displayed on a user interface of a user device, such as any of the user devices 104 of FIG. 1.

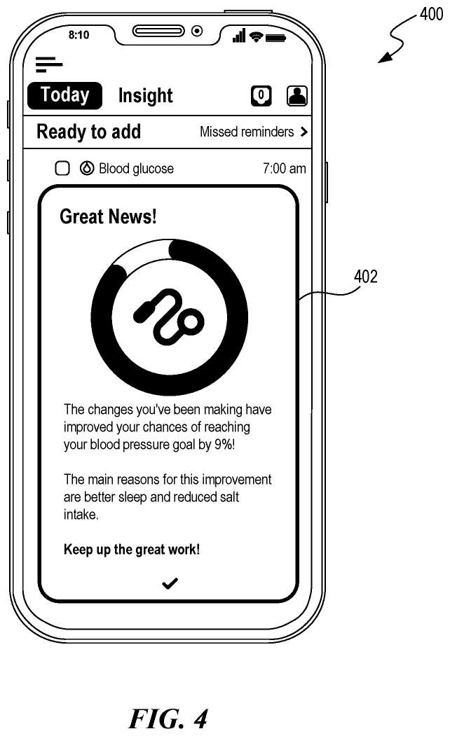

FIG. 4 illustrates a representative example of a user interface 400 including a graphical representation 402 of a support message configured in accordance with embodiments of the present technology. In the illustrated embodiment, the support message indicates the predicted probability of reaching the user's health metric goal (“improved your chances of reaching your blood pressure goal by 9%”) an explanation of key health factors contributing to the prediction (“The main reasons for this improvement are better sleep and reduced salt intake”), and recommended actions (“Keep up the great work!”).

Referring again to FIG. 3C, at block 332, the process 304 can optionally include obtaining user feedback. In some embodiments, a suitable system (e.g., the system 102 of FIG. 1) may be configured to collect user feedback that may be entered using a user device (e.g., any of the user devices 104 of FIG. 1). The user feedback may be indicative of whether the presented message was useful, not useful, etc. (e.g., “Helpful,” “Not Helpful,” etc.). Further data collected by the user device (e.g., through an appropriately installed application) may be used to determine whether the user is acting in a way suggested in the support message. For example, if a support message recommends reducing salt intake, subsequent logged food entries may be monitored and evaluated to determine whether salt consumption was indeed reduced. Moreover, selection of future support messages may be adjusted based on the received user feedback, as well as generated forecasts.

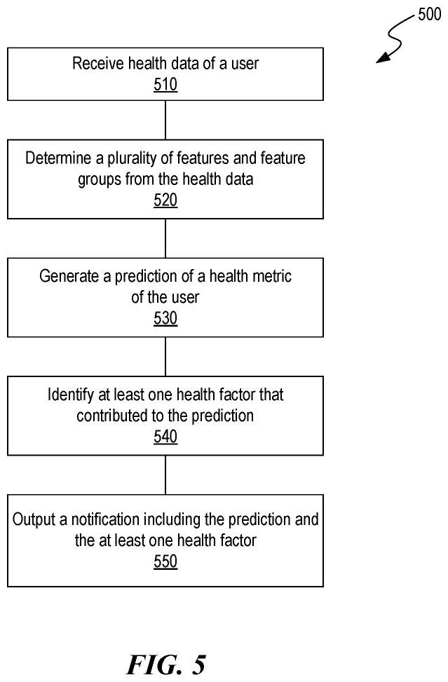

FIG. 5 is a flowchart illustrating a method 500 for forecasting a health metric of a user, in accordance with embodiments of the present technology. The method 500 can be performed by any of the systems and devices described herein. For example, some or all of the steps of the method 500 can be performed by the system 102 and/or the user devices 104 of FIG. 1. In some embodiments, the method 500 is performed by a computing system or device including one or more processors and a memory storing instructions that, when executed by the one or more processors, cause the computing system or device to perform one or more of the steps described herein. The method 500 can be combined with any of the other methods or processes described herein, such as the processes 300, 302, 304 of FIGS. 3A-3C.

The method 500 can be used to predict any of the health metrics described herein. For example, the health metrics can include metrics related to any of the following: blood pressure, blood glucose (e.g., 30-day time-in-range and/or other time-in-range metric), weight, BMI, waist circumference, body fat percentage, cholesterol, triglycerides, heart rate, respiratory rate, body temperature, gases (e.g., oxygen, carbon dioxide), electrolytes (e.g., bicarbonate, potassium, sodium, magnesium, chloride, lactic acid), BUN, creatinine, ketones, alcohols, neurotransmitters, amino acids, hormones, disease biomarkers (e.g., cancer biomarkers, cardiovascular disease biomarkers), and/or combinations thereof. The prediction can be made for any suitable time period, such as at least one week, two weeks, three weeks, four weeks, one month, two months, three months, four months, five months, six months, seven months, eight months, nine months, ten months, 11 months, or 12 months in the future from the date of the prediction.

At block 510, the method 500 begins with receiving health data of a user. As discussed above, the health data can include any data relevant to the user's health state. Examples of health data include, but are not limited to, any of the following: blood pressure data (e.g., current and/or previous measurements of systolic and/or diastolic blood pressure), blood glucose data (e.g., current and/or previous blood glucose measurements, current and/or previous HbA1c data values), heart rate data, food data (e.g., number of meals; timing of meals; number of calories; amount of carbohydrates, fats, sugars, etc.), medical history data (e.g., current and/or previous weight, height, BMI, age, sleeping patterns, medical conditions, cholesterol levels, diabetes type, family history, user health history, diagnoses, blood pressure, etc.), activity data (e.g., time and/or duration of activity; activity type such as walking, running, swimming; strenuousness of the activity such as low, moderate, high; etc.), personal data (e.g., name, gender, demographics, social network information, etc.), medication data (e.g., timing and/or dosages of medications such as insulin, prescription and/or non-prescription medications taken), and/or any other suitable data (e.g., basal energy consumption, oxygen consumption) or combination thereof. The health data can be obtained automatically by one or more biosensors (e.g., the biosensor(s) 104a of FIG. 1), input manually by the user (e.g., via the user devices 104 of FIG. 1), retrieved from a database and/or server (e.g., database 106 of FIG. 1), or any other suitable collection technique.

Optionally, block 510 can further include receiving a health goal for the user. The health goal can be a target value and/or range for a particular health metric (e.g., blood pressure, blood glucose, weight, etc.) that the user wishes to achieve in the future (e.g., one, two, three, four, five, six, or more months in the future). For example, the health goal can be for the user's health metric to achieve a target value and/or range, to be greater than a target value and/or range, to be less than a target value and/or range, etc. The health goal can be determined by the user, by a healthcare professional, set based on healthcare guidelines (e.g., based on the user's characteristics), or suitable combinations thereof. The health goal can be input by the user via a user device, transmitted to the system from a healthcare professional's device, retrieved from a database or server, or any other suitable technique. Optionally, block 510 can include receiving multiple health goals for multiple different health metrics.

At block 520, the method 500 continues with determining a plurality of features and feature groups from the health data of block 510. The features can be determined from the health data using any suitable approach. For example, as discussed above with reference to FIGS. 3A-3C, the features can include values (e.g., the most recent value or set of values) and/or statistics (e.g., averages, standard deviations, ranges, sums, differences, ratios, maximums, minimums, percentiles, probabilities, cross-correlations, time-in-range values) for any suitable health data, including, but not limited to, any of the following: blood glucose levels, blood pressure levels, heart rate, weight, food intake, medical history, demographics, diagnoses and/or medical conditions, medications, sleep patterns, activity patterns, and/or combinations thereof. Features can be computed across health data obtained over any suitable length of time (e.g., 15 days, 30 days, 60 days, or 90 days) and at any suitable time period before the prediction is made (e.g., immediately before the prediction, 15 days before the prediction, 30 days before the prediction, 60 days before the prediction, or 90 days before the prediction).

In some embodiments, the features are classified in a plurality of feature groups, each feature group being associated with a respective health factor. Each health factor can relate to an aspect of the user's health that may influence the predicted health metric. As previously discussed with reference to FIGS. 3A-3C, examples of health factors that may be used to determine feature groups include, but are not limited to: blood pressure, blood glucose, heart rate, multi-factor interactions, demographic factors, meal intake, sleep, weight, activity, medical conditions and/or diagnoses, and the like. Each feature in a particular feature group may be derived from measurements and/or other data for the corresponding health factor. In some embodiments, the features can be categorized into at least one, two, three, four, five, ten, or more different feature groups.

At block 530, the method 500 can include generating a prediction of a health metric of the user. The prediction can be generated using one or more prediction models, e.g., as previously discussed with reference to FIGS. 3A-3C. For example, the prediction can be generated by inputting at least some of the features determined in block 520, and, optionally, at least some of the health data received in block 510, into the prediction model(s). In some embodiments, the prediction model(s) are or include one or more machine learning models (e.g., a Gradient Boosted Trees model). In such embodiments, the machine learning model(s) can be trained on health data from a plurality of different users, e.g., in accordance with the techniques described above with reference to FIGS. 3A and 3B. The training data may include data for the particular user for which the prediction is to be generated, or may not any include any data from the particular user. As previously discussed, the use of training data from a large number of users allows accurate predictions to be made even for users with limited, irregular, and/or incomplete health data.

The prediction can provide an estimated value and/or range for the health metric at a future time point. The future time point can be at least one, two, three, four, five, six, seven, eight, nine, ten, 11, 12, or more months from the date of the prediction. Alternatively or in combination, the prediction can provide an estimated probability that the health metric will achieve a particular target value and/or range at the future time point. In such embodiments, the predicted probability can be expressed quantitatively (e.g., an x % chance of achieving the goal) and/or qualitatively (e.g., highly likely, likely, unlikely, highly unlikely).

At block 540, the method 500 can also include identifying at least one health factor that contributed to the prediction. The health factor can be identified in various ways, such as by selecting one or more feature groups that provided a threshold contribution to the prediction, then determining the health factor(s) associated with the selected feature group(s). For example, as previously discussed with reference to FIGS. 3A-3C, the contribution of at least some of the features used in step 520 and 530 can be determined using an attribution algorithm (e.g., a SHAP algorithm) or other suitable technique. The attribution algorithm can be configured to calculate a quantitative value (e.g., a marginal contribution value) representing the contribution of each feature to the prediction of the health metric. Subsequently, the contributions of each feature within a feature group can be aggregated (e.g., summed) to generate a subtotal representing the net marginal contribution of that particular feature group. The magnitude of the contribution of each feature group can correlate to the influence of that feature group on the final prediction, e.g., a larger magnitude can indicate a more influential feature group, a smaller magnitude can indicate a less influential feature group, etc.

Based on the determined contributions, block 540 can further include identifying one or more feature groups determined to have contributed to the prediction (e.g., feature group(s) whose contribution met a threshold value and/or other suitable criteria). For example, block 540 can include identifying at least one, two, three, or more feature groups having the greatest contribution(s) to the prediction, e.g., by ranking the feature groups in order of contribution magnitude. As another example, feature groups can be identified based on the percentage and/or proportion of the contribution made to the prediction, e.g., all feature groups contributing at least 10%, 25%, 50%, or 75% to the prediction; feature groups that collectively account for at least 50%, 75%, 90%, or 95% of the prediction; and so on.

At block 550, the method 500 can include outputting a notification to the user. The notification can include the prediction of the health metric and, optionally, the at least one health factor determined to have contributed to the prediction (e.g., as discussed above with reference to block 540). For example, if the blood glucose feature group was determined to have contributed to the prediction, the notification can include a support message or other feedback informing the user that the predicted outcome can be at least partially attributed to the user's blood glucose levels. The notification can be provided in any suitable format, such as textual, visual, graphical, audible, and/or other formats. The notification can be output to the user via a graphical user interface on a user device or any other suitable computing device.

The method 500 can optionally include determining a recommended action for the user to improve their health metrics, based on the prediction and/or contributing health factor(s). For example, if the method 500 determines that a certain health factor is particularly influential in causing the user to achieve or not achieve their health goal, the notification can inform the user with recommended actions with respect to that health factor (e.g., decreasing blood glucose levels; increasing physical activity; improving sleep patterns; altering dietary intake; etc.). The recommendation can be customized to the particular user, e.g., based on user feedback, behavioral patterns, etc. For example, the method 500 can account for whether the user has historically complied or not complied with a particular lifestyle change, whether the user has expressed a preference for certain types of behavioral interventions, etc. Such information can also be used as input for generating future recommendations and/or other notifications to assist the user in meeting their health goals.

In some embodiments, the approaches described herein provide improved accuracy in predicting various health metrics (e.g., 30-day time-in-range, 30-day average blood glucose values, 30-day average blood pressure values, weight, etc.) compared to conventional techniques. For example, the RMSE of predictions generated in accordance with embodiments of the present technology can be no more than 20%, 15%, 10%, or 5%. The relative reduction in RMSE compared to a naïve predictor (e.g., a persistence model) can be at least 10%, 15%, 20%, 25%, 30%, 40%, or 50%. The enhanced accuracy can assist users in monitoring and managing their health conditions, as well as in making decisions regarding lifestyle changes and/or other actions to improve their health.

### Additional Embodiments

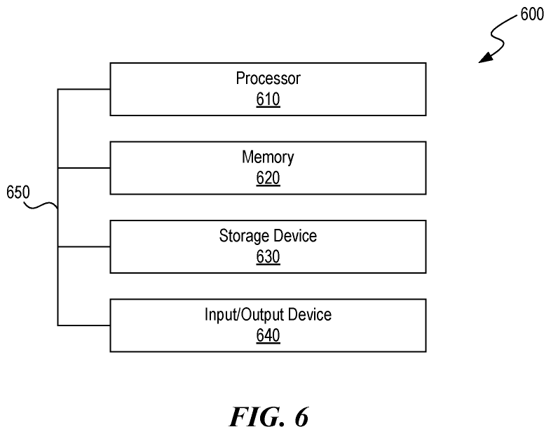

FIG. 6 is a schematic block diagram of a computing system or device (“system 600”) configured in accordance with embodiments of the present technology. The system 600 can be incorporated into or used with any of the systems and devices described herein, such as the system 102 and/or user devices 104 of FIG. 1. The system 600 can be used to perform any of the processes or methods described herein with respect to FIGS. 1-5. The system 600 can include a processor 610, a memory 620, a storage device 630, and an input/output device 640. Each of the components 610, 620, 630 and 640 can be interconnected using a system bus 650. The processor 610 can be configured to process instructions for execution within the system 600. In some embodiments, the processor 610 is a single-threaded processor. Alternatively, the processor 610 can be a multi-threaded processor. The processor 610 can be further configured to process instructions stored in the memory 620 or on the storage device 630, including receiving or sending information through the input/output device 640. The memory 620 can store information within the system 600. In some embodiments, the memory 620 is a computer-readable medium. Optionally, the memory 620 can be a volatile memory unit or a non-volatile memory unit. The storage device 630 can be capable of providing mass storage for the system 600. In some embodiments, the storage device 630 is a computer-readable medium. Optionally, the storage device 630 can be a floppy disk device, a hard disk device, an optical disk device, a tape device, non-volatile solid state memory, or any other type of storage device. The input/output device 640 can be configured to provide input/output operations for the system 600. In some embodiments, the input/output device 640 can include a keyboard and/or pointing device. The input/output device 640 can also include a display unit for displaying graphical user interfaces.

The systems and methods disclosed herein may be embodied in various forms including, for example, a data processor, such as a computer that also includes a database, digital electronic circuitry, firmware, software, or in combinations thereof. Moreover, the above-noted features and other aspects and principles of the present disclosed implementations may be implemented in various environments. Such environments and related applications may be specially constructed for performing the various processes and operations according to the embodiments disclosed herein, or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the disclosed embodiments, or it may be more convenient to construct a specialized apparatus or system to perform the required methods and techniques.

The systems and methods disclosed herein may be implemented as a computer program product, i.e., a computer program tangibly embodied in an information carrier, e.g., in a machine readable storage device or in a propagated signal, for execution by, or to control the operation of, data processing apparatus, e.g., a programmable processor, a computer, or multiple computers. A computer program may be written in any form of programming language, including compiled or interpreted languages, and it may be deployed in any form, including as a stand-alone program or as a module, component, subroutine, or other unit suitable for use in a computing environment. A computer program may be deployed to be executed on one computer or on multiple computers at one site or distributed across multiple sites and interconnected by a communication network.

These computer programs, which may also be referred to programs, software, software applications, applications, components, or code, include machine instructions for a programmable processor, and may be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium may store such machine instructions non-transitorily, such as for example as would a non-transient solid state memory or a magnetic hard drive or any equivalent storage medium. The machine-readable medium may alternatively or additionally store such machine instructions in a transient manner, such as for example as would a processor cache or other random access memory associated with one or more physical processor cores.

To provide for interaction with a user, the subject matter described herein may be implemented on a computer having a display device, such as for example a cathode ray tube (CRT) or a liquid crystal display (LCD) monitor for displaying information to the user and a keyboard and a pointing device, such as for example a mouse or a trackball, by which the user may provide input to the computer. Other kinds of devices may be used to provide for interaction with a user as well. For example, feedback provided to the user may be any form of sensory feedback, such as for example visual feedback, auditory feedback, or tactile feedback; and input from the user may be received in any form, including, but not limited to, acoustic, speech, or tactile input.

The technology described herein may be implemented in a computing system that includes a back-end component, such as for example one or more data servers, or that includes a middleware component, such as for example one or more application servers, or that includes a front-end component, such as for example one or more client computers having a graphical user interface or a Web browser through which a user may interact with an embodiment of the technology described herein, or any combination of such back-end, middleware, or front-end components. The components of the system may be interconnected by any form or medium of digital data communication, such as for example a communication network. Examples of communication networks include, but are not limited to, a local area network (“LAN”), a wide area network (“WAN”), and the Internet.

The computing system may include clients and servers. A client and server are generally, but not exclusively, remote from each other and typically interact through a communication network. The relationship of client and server arises by virtue of computer programs running on the respective computers and having a client-server relationship to each other.

### CONCLUSION

The embodiments set forth in the foregoing description do not represent all embodiments consistent with the subject matter described herein. Instead, they are merely some examples consistent with aspects related to the described subject matter. Although a few variations have been described in detail above, other modifications or additions are possible. In particular, further features and/or variations may be provided in addition to those set forth herein. For example, the embodiments described above may be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other embodiments may be within the scope of the following claims.

As used herein, the term “user” may refer to any entity including a person or a computer.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

As used herein, the phrase “and/or” as in “A and/or B” refers to A alone, B alone, and A and B.

As used herein, the term “user” can refer to any entity including a person or a computer.

Although ordinal numbers such as first, second, and the like can, in some situations, relate to an order; as used in this document ordinal numbers do not necessarily imply an order. For example, ordinal numbers can be merely used to distinguish one item from another. For example, to distinguish a first event from a second event, but need not imply any chronological ordering or a fixed reference system (such that a first event in one paragraph of the description can be different from a first event in another paragraph of the description).

From the foregoing, it will be appreciated that specific embodiments of the invention have been described herein for purposes of illustration, but that various modifications may be made without deviating from the scope of the invention. Accordingly, the invention is not limited except as by the appended claims.

I/We claim:

1. A computer-implemented method for informing a user of at least one health factor contributing to a predicted health metric, the method comprising:
receiving health data of the user;
calculating a plurality of features based on the health data, wherein the features are categorized into a plurality of feature groups;
generating a prediction of a health metric of the user by inputting the features into a machine learning model;
identifying at least one contributing health factor by:
selecting at least one feature group that provides a threshold contribution to the prediction, and
determining a health factor associated with the at least one feature group; and
outputting a notification for the user including the prediction and the at least one contributing health factor.

2. The computer-implemented method of claim 1 wherein:
the health data includes one or more of blood pressure data, blood glucose data, heart rate data, food data, activity data, sleep data, weight data, medication data, diagnosis data, or demographics data;
the features include one or more of values or statistics calculated from the health data;
the prediction of the health metric includes a predicted future value or range for a blood pressure level of the user; and
the at least one contributing health factor includes one or more of blood pressure, blood glucose, heart rate, food, activity, sleep, weight, medication, diagnosis, or demographics.

3. The computer-implemented method of claim 1 wherein selecting the at least one feature group comprises:
calculating an individual contribution of at least some of the features to the prediction; and
for each feature group,
summing the individual contributions of each feature within the feature group to calculate the contribution of the feature group, and
selecting the feature group if the contribution meets a threshold value.

4. The computer-implemented method of claim 3 wherein the contribution of each feature group is determined using an attribution algorithm.

5. The computer-implemented method of claim 4 wherein the attribution algorithm includes a Shapley Additive Explanations (SNAP) algorithm.

6. The computer-implemented method of claim 1 wherein selecting the at least one feature group comprises selecting the feature group having the largest contribution to the prediction.

7. The computer-implemented method of claim 1 wherein selecting the at least one feature group comprises selecting a plurality of feature groups that collectively provide the threshold contribution to the prediction.

8. The computer-implemented method of claim 1 wherein the notification includes a recommended action for the user related to the at least one contributing health factor.

9. The computer-implemented method of claim 1 wherein the prediction includes a value or range for the health metric at a future time period.

10. The computer-implemented method of claim 9 wherein the future time period is at least one month in the future.

11. The computer-implemented method of claim 1, further comprising receiving an indication of a goal for the health metric, wherein the prediction includes a probability of achieving the goal.

12. The computer-implemented method of claim 11, wherein the goal is set by the user.

13. The computer-implemented method of claim 1 wherein the machine learning model is trained on health data from a plurality of different users.

14. A system for predicting a health metric of a user, the system comprising:
one or more processors; and
a memory storing instructions that, when executed by the one or more processors, cause the system to perform operations comprising:
receiving a plurality of health measurements for the user;
determining a set of features based on the health measurements, wherein the features are classified into a set of feature groups, each feature group being associated with a health factor;
generating a prediction of the health metric by inputting the features into a machine learning model, wherein the prediction includes a probability that the health metric will reach a target value or range at a future time period;
identifying at least one feature group that provided a threshold contribution to the prediction; and
outputting a notification for the user including the prediction and an indication of the at least one feature group.

15. The system of claim 14 wherein the set of feature groups includes feature groups associated with one or more of the following health factors: blood pressure, blood glucose, demographics, diagnoses, medications, sleep, food, weight, or activity.

16. The system of claim 14 wherein identifying the at least one feature group comprises:
computing an individual contribution of at least a subset of features to the prediction; and
for each feature group, summing the individual contributions of each feature within the feature group to calculate the contribution of the feature group.

17. The system of claim 15 wherein the contribution of each feature group is determined using an attribution algorithm.

18. The system of claim 14 wherein the at least one feature group includes a feature group having the largest contribution to the prediction.

19. The system of claim 14 wherein the health metric includes one or more of the following: blood pressure level, blood glucose level, weight, body mass index, waist circumference, cholesterol level, or triglycerides level.

20. The system of claim 14 wherein the future time period is at least three months in the future.

21. The system of claim 14, further comprising a biosensor operably coupled to the one or more processors, wherein the biosensor is configured to generate at least some of the health measurements.

22. The system of claim 21 wherein the biosensor includes a blood pressure sensor or a blood glucose sensor.

23. The system of claim 14 wherein the operations further comprise transmitting the notification to a user device.

24. The system of claim 23 wherein the user device includes a wearable device or a mobile device.

25. A non-transitory computer-readable storage medium including instructions that, when executed by a computing system, cause the computing system to perform operations comprising:
receiving health data of a user, wherein the health data includes measurements for a plurality of health factors;
determining a plurality of features from the health data, wherein the features are categorized into a plurality of feature groups, each feature group being associated with a corresponding health factor;
generating a prediction of a health metric of the user at a future time point by inputting the features into a machine learning model;
determining a contribution of each feature group to the prediction; and
outputting a notification to the user including the prediction and a health factor associated with a feature group having the largest contribution to the prediction.
